(19) United States
(12) Patent Application Publication
(10) Pub. No.: US 2023/0218757 A1
(43) Pub. Date: Jul. 13, 2023

(54) AUGMENTATION OF FIBROBLAST THERAPY USING EXTRACORPOREAL SHOCK WAVE THERAPY AND/OR TRANSFECTION OF BIOLOGICALLY RELEVANT MOLECULES

(71) Applicant: FIGENE, LLC, Houston, TX (US)

(72) Inventor: Thomas ICHIM, San Diego, CA (US)

(21) Appl. No.: 17/996,102

(22) PCT Filed: Apr. 16, 2021
(86) PCT No.: PCT/US2021/027654

§ 371 Date: Oct. 13, 2022

Related U.S. Application Data
(60) Provisional application No. 63/012,200, filed on Apr. 19, 2020.

(51) Int. Cl. A61K 41/00; A61K 35/33; A61N 7/00; A61K 45/06; A61K 35/28; C12N 5/077

(52) U.S. Cl. CPC A61K 41/0047; A61K 35/33; A61N 7/00; A61K 45/06; A61K 35/28; C12N 5/0656; A61K 2035/124

(57) ABSTRACT

Disclosed are means of enhancing therapeutic effects of fibroblast administration through utilization of extracorporeal shock waves. In one embodiment, enhancement of intravenously administered fibroblast therapeutic activity is accomplished by introducing extracorporeal shock waves to the patient in need of therapy. In one specific embodiment, enhancement of the ability of fibroblasts administered intravenously to treat a condition is accomplished by exposure of areas areas affected by the condition to extracorporeal shock waves. In another specific embodiment, the invention provides transfection of IL-12 and/or IL-23 into fibroblasts to augment regenerative activity, including neuroregenerative and anticancer activity. In further embodiments the invention provides augmentation of regenerative activity by induction of T regulatory cells utilizing IL-35 transfection, wherein said T regulatory cells provide an optimized environment for stimulation of regenerative activity.

This application claims priority to U.S. Provisional Patent Application Ser. No. 63/012,200, filed Apr. 19, 2020, which is incorporated by reference herein in its entirety.

### TECHNICAL FIELD

Embodiments of the disclosure generally include at least the fields of cell biology, molecular biology, sonography, and medicine. More particularly, the disclosure pertains at least to the area of enhancing the effects of therapeutic fibroblast administration through utilization of extracorporeal shock waves.

### BACKGROUND

Extracorporeal shockwave therapy (“ESWT”) is non-surgical, non-invasive treatment of medical conditions using acoustic shockwaves induced by ultrasound. Shockwave therapy was first used in the early 1980's to fragment kidney stones, termed shockwave lithotripsy, such that stones previously manageable only by invasive techniques could be passed from the urinary tract. To fragment kidney stones, the ESWT is delivered as a 30- to 45-minute treatment that yields a 50% to 75% stone-free rate at three months after the procedure. Medical literature has suggested that lithotripsy can cause hypertension and kidney damage, including hematuria, and hemorrhage during the procedure, though newer technologies have reduced this risk. Specifically, induction of pathological cavitation by shockwaves must be reduced to avoid these adverse effects. Use of ESWT has also been applied to gall bladder stones.

The use of ESWT for pathologies besides stones was first introduced in studies assessing the efficacy of ESWT in Peyronie's disease, in which shockwaves were believed to contribute to degradation of penile plaques. Continued development of ESWT has since resulted in a variety of other possible applications including calcification fragmentation, chronic orthopedic inflammation healing, bone healing and stimulation of bone formation (osteogenesis), wound healing, revascularization, angiogenesis stimulation bone of formation, and angiogenesis. As an example, ESWT has been used for invasive stimulation of the kidneys by surgically exposing organs, for example the heart or kidneys, prior to applying shockwave therapy, as described in U.S. Pat. No. 7,507,213. Another example of ESWT includes use of high-intensity focused ultrasound (HIFU) to ablate innervated kidney tissue, denervate renal vasculature, including disruption and termination of renal sympathetic nerve activity, and improve cardiac and/or renal function, particularly that associated with hypertension, as described in U.S. Patent Pub. No. 2011/0257523.

Further studies have suggested that ESWT could be effective in regenerative medicine. In one clinical trial, 208 patients with complicated, non-healing, acute and chronic soft-tissue wounds were treated with debridement, outpatient ESWT, and moist dressings. The outpatient ESWT as comprised of 100 to 1000 shocks/cm² at 0.1 mJ/mm², according to wound size, every 1 to 2 weeks over mean three treatments. Of 208 patients enrolled, 156 (75%) had 100% wound epithelialization. During a mean follow-up period of 44 days, no patients exhibited treatment-related toxicity, infection, or deterioration of any ESWT-treated wound. Intent-to-treat multivariate analysis identified age (P=0.01), wound size < or =10 cm² (P=0.01; OR=0.36; 95% CI, 0.16 to 0.80), and duration < or =1 month (P<0.001; OR=0.25; 95% CI, 0.11 to 0.55) as independent predictors of complete healing. Subsequent studies have demonstrated the potential regenerative activity of ESWT in other, diverse areas including heart failure, non-union bone fractures, limb ischemia, ulcer healing, and spinal cord injury.

Shockwaves, a form of acoustic energy, are applied therapeutically in ESWT. Shockwaves result from phenomena that create a sudden intense change in pressure, for example, an explosion or lightning. Shock waves have different characteristics in comparison to ultrasound. Ultrasound (US) usually consists of periodic oscillations with limited bandwidth. The effects of US on tissue can generally be divided into thermal and non-thermal mechanisms. Absorption of US waves by tissue leads to increased local temperatures. While diagnostic US leads to low or negligible increases in tissue temperature, therapeutic US involves increasing the pulse length or power of US waves to generate rapid heating, coagulation, liquefactive necrosis, tissue vaporization, or a combination thereof. Therapeutic US is typically harnessed for applications that require tissue necrosis, such as ablation of soft tissue tumors, and, in cardiovascular applications for treatment of cardiac arrhythmias.

Conversely, shock waves are single, mainly positive pressure pulses that are followed by comparatively small tensile wave components. Ultrasound applies an alternating high frequency load to the tissue, with a frequency range of several megahertz, thus leading to heating, tissue tears and cavitation at high amplitudes. The shock wave's effect in comparison, is forward directed energy (in the direction of the shock wave propagation). Its force takes effect at the interface and further that can be increased to enable the destruction of kidney stones, for instance. Since these dynamic effects basically occur at interfaces with a jump in the acoustic resistance but hardly ever in homogenous media (tissue, water), shock waves are the ideal means for creating effects in deep tissue without affecting the tissue in front of it.

The application of shock waves, particularly therapeutic and diagnostic approaches, relies on piezoelectric crystals which, when electrically stimulated, release high frequency sound waves. As these sound waves propagate through tissue, their energy is partially absorbed and partially reflected by fluid, cells, and connective tissue. Based on the clinical application and tissue characteristics, parameters including frequency, amplitude, and pulse duration can be optimized to maximize reflection and minimize absorption for diagnostic imaging or to minimize reflection and maximize absorption for therapeutic applications.

Unlike therapeutic US waves, shockwaves do not produce heat within the tissue. While some low amplitude ultrasound waveforms produce a constant cyclic sinusoidal amplitude that generates heat at the tissue level, shockwaves do not have constant amplitude over time. Specifically, shockwaves are characterized by the delivery of a sequence of transient pressure disturbances characterized by an initial high peak pressure and amplitude with a rapid pressure rise followed by rapid wave propagation with amplitude diminishing to 10-20% of the initial pressure peak over the wave lifecycle. These intense changes in pressure produce strong waves of energy that can travel through any elastic medium, such as air, water, human soft tissue, or certain solid substances like bone.

Relatively high acoustic amplitude shockwaves can potentiate non-thermal effects. The pressure of the shockwave causes cavitation, the creation of small gas bubbles in the blood via transmission of energy, and collapse of the cavitation bubbles is in part responsible for the efficacy of the therapy. The waves are focused in order to achieve the effects in a volume limited zone of tissue, typically several mm across (2-8 mm), and thus the destructive effects of shockwaves are eliminated. Acoustic cavitation mediates the vascular effects of shockwaves through three potential mechanisms. One of these mechanisms is microstreaming, which is the transmission of shear stress from fluid motion generated by the oscillation of bubbles against cell membranes and endothelial surfaces. US waves may also directly induce shear stress onto cell membranes. Another mechanism is jetting from asymmetric collapse of a bubble. Jetting of bubbles from vessel lumen into tissue, known as poking, or from tissue into the lumen leading to invagination of the local vessel wall can potentially increase vascular permeability. Permeability may also be enhanced by expanding and compressing bubbles, which, in turn, causes distension and invagination of vessel walls, respectively.

Acoustic shockwaves are primarily generated electro-hydraulically (spark gap), electromagnetically (“EMSE”), and piezo-electrically. Each of the three methods requires an apparatus to focus the generated shockwave so as to provide a focal point and/or focal zone for the treatment area. Shockwaves produce much higher pressure impulses within the focal zone as compared with zones outside the focal zone. The waves produced by the three methods can be mechanically focused by an appropriate arrangement of surfaces reflecting the wave toward the desired focal point and/or an appropriate arrangement of the generating devices. The method of focusing the generated shockwave has been extensively described in the art, for example in U.S. Pat. Nos. 5,174,280, 5,058,569, and 5,033,456, and EP1591070, all of which are incorporated herein by reference. These patents cover various systems for generating focused shockwaves, suitable for extracorporeal therapies. These systems have substantially hollow, cylindrical membranes consisting of electrically conductive materials and electrical coil arrangements disposed inside the membrane which can be supplied with a high voltage pulse to rapidly repel the membrane and thereby generate shockwaves, which can be focused.

EMSE systems utilize an electromagnetic coil to initiate a shockwave and an opposing metal membrane for focusing. Specifically, in electromagnetic systems, an electrical impulse is circulated in a coil. The coil produces an electromagnetic field that expels a metallic membrane to produce the mechanical impulse.

Piezoelectric systems form acoustical waves using piezoelectric crystals which are mounted to a spherical surface to provide focus. Specifically, in piezoelectric systems, ceramic material with piezoelectric characteristics is subjected to an electrical impulse. The electric impulse modifies the dimension of the ceramic material to generate the desired mechanical impulse. A focal point is attained by covering a concave spherical surface with piezoelectric ceramics converging at the center of the sphere.

Electro-hydraulic, or spark gap, systems incorporate an electrode (spark plug), to initiate a shockwave, and an ellipsoid to focus the shockwave. In spark gap systems, high energy shockwaves are generated by applying electricity to an electrode positioned in an ellipsoid immersed in treated water. When the electrical charge is fired, a small amount of water is vaporized at the tip of the electrode, and a shockwave is produced. The shockwave ricochets from the side of the ellipsoid and converges at a focal point, which may then be transferred to the area to be treated.

The augmentation of effects of fibroblast-mediated regeneration by shockwaves has not been investigated. There is a need for using ESWT to enhance the therapeutic efficacy and activity of regenerative fibroblasts to modulate biological systems by stimulating regeneration, providing immune system modulation, suppressing inflammation, and stimulating angiogenesis, and to use ESWT to improve the efficacy of gene-transfected fibroblasts.

### BRIEF SUMMARY

The present disclosure, in some embodiments, is directed to methods and systems related to stimulating regeneration of cells and/or tissue in an individual using extracorporeal shockwave therapy and fibroblast cells.

Disclosed herein are methods for stimulating regeneration of cells or tissue in an individual, comprising the step of administering an extracorporeal shockwave therapy (ESWT) regimen and fibroblast cells to an anatomical area in an individual in need of regeneration. Also disclosed herein are systems comprising ESWT administered together with fibroblast cells.

In some embodiments of the methods and systems, the fibroblasts comprise regenerative fibroblasts. In some embodiments, the fibroblast cells are cultured under conditions sufficient to differentiate the fibroblasts into regenerative fibroblast cells. The regenerative fibroblast cells can comprise one or more of the following biological activities: (a) inducing of angiogenesis; (b) modulating the immune system; (c) suppressing inflammation; (d) preventing of tissue atrophy; (e) regenerating of functional tissue; (f) inhibition of neuronal cell dysfunction; and (g) inhibition of smooth muscle degeneration.

In some embodiments, the regenerative fibroblast cells are cultured under conditions sufficient to enhance the ability of the regenerative fibroblast cells to induce angiogenesis, prevent tissue atrophy, regenerate functional tissue, inhibit neuronal cell dysfunction, inhibit smooth muscle degeneration, or a combination thereof. The conditions can comprise hypoxia. The conditions can further comprise treatment of the regenerative fibroblast cells with one or more growth factors, one or more differentiation factors, one or more dedifferentiation factors, or a combination thereof.

In some embodiments, the regenerative fibroblast cells express one or more markers selected from the group consisting of Oct-4, Nanog, Sox-2, KLF4, c-Myc, Rex-1, GDF-3, LIF receptor, CD105, CD117, CD344, Stella, and a combination thereof. In some embodiments, the regenerative fibroblast cells do not express one or more cell surface proteins selected from the group consisting of MHC class I, MHC class II, CD45, CD13, CD49c, CD66b, CD73, CD105, CD90, and a combination thereof. In some embodiments, the regenerative fibroblast cells have enhanced GDF-11 expression compared to a control or standard.

In some embodiments of the methods and systems, the fibroblast cells are, or are derived from, fibroblasts isolated from umbilical cord, skin, cord blood, adipose tissue, hair follicle, omentum, bone marrow, peripheral blood, Wharton's Jelly, or a combination thereof. In some embodiments, the fibroblast cells are dermal fibroblasts, placental fibroblasts, adipose fibroblasts, bone marrow fibroblasts, foreskin fibroblasts, umbilical cord fibroblasts, hair follicle derived fibroblasts, nail derived fibroblasts, endometrial derived fibroblasts, keloid derived fibroblasts, or a combination thereof. The fibroblast cells may be autologous, allogeneic, or xenogeneic to the recipient. In some embodiments, the fibroblast cells are purified from bone marrow.

In some embodiments, the fibroblast cells are purified from peripheral blood. In some embodiments, the regenerative fibroblast cells are isolated from peripheral blood of an individual who has been exposed to one or more conditions and/or one or more therapies sufficient to stimulate regenerative fibroblast cells from the individual to enter the peripheral blood of the individual. The conditions sufficient to stimulate regenerative fibroblast cells from the individual to enter the peripheral blood can comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof. The therapies sufficient to stimulate regenerative fibroblast cells from the individual to enter the peripheral blood can comprise therapies including exercise, hyperbaric oxygen, autohemotherapy by ex vivo ozonation of peripheral blood, induction of SDF-1 secretion in an anatomical area outside of the bone marrow, or a combination thereof.

In some embodiments, the regenerative fibroblast cells are comprised of an enriched population of regenerative fibroblast cells. In some embodiments, enrichment is achieved by: (a) transfecting the cells with a vector comprising a promoter, including in some cases a fibroblast-specific promoter, operably linked to a reporter or selection gene, wherein the reporter or selection gene is expressed, and (b) enriching the population of cells for cells expressing the reporter or selection gene. In some embodiments, enrichment is achieved by: (a) treating the cells with a detectable compound, wherein the detectable compound is selectively detectable in proliferating and non-proliferating cells, and (b) enriching the population of cells for proliferating cells. The detectable compound can be selected from the group consisting of carboxyfluorescein diacetate, succinimidyl ester, Aldefluor, and a combination thereof.

In some embodiments, the regenerative fibroblast cells are fibroblasts isolated as side population cells. The fibroblasts isolated as side population cells can be identified based on expression of the multidrug resistance transport protein (ABCG2) and/or based on the ability to efflux intracellular dyes. The side population cells may be derived from tissues selected from the group consisting of pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, and a combination thereof.

In some embodiments, the fibroblast cells express CD73. The CD73-positive fibroblast cells can be derived from the group consisting of foreskin, adipose tissue, skin biopsy, bone marrow, placenta, umbilical cord, placenta, umbilical cord blood, ear lobe skin, and a combination thereof. The CD73-positive fibroblast cells are cultured under hypoxic conditions. In some embodiments, the hypoxic conditions comprise from 0.1% oxygen to 10% oxygen for a period of 30 minutes to 3 days. In some embodiments, the hypoxic conditions comprise 3% oxygen for 24 hours. In some embodiments, hypoxic conditions are chemically induced. Chemical induction of hypoxia can comprise culture in cobalt (II) chloride. In some embodiments, fibroblast cells are cultured with 1 μM-300 μM cobalt (II) chloride. In some embodiments, the fibroblast cells are incubated with 250 μM of cobalt (II) chloride. In some embodiments, the fibroblast cells are further cultured for 1-48 hours. In some embodiments, the fibroblast cells are cultured for a time period of 24 hours. In some embodiments, the hypoxic conditions induce upregulation of HIF-1α. Expression of HIF-1α can be detected by expression of VEGF secretion. In some embodiments, the hypoxic conditions induce upregulation of CXCR4 on the fibroblast cells. Upregulation of CXCR4 can promote homing of the fibroblast cells to an SDF-1 gradient.

In some embodiments of the methods and systems, CD73-positive fibroblast cells are cultured under conditions to suppress expression of one or more apoptosis-associated genes. The one or more apoptosis-associated genes can be selected from the group consisting of Fas, FasL, CASP1 (ICE), CASP10 (MCH4), CASP14, CASP2, CASP3, CASP4, CASPS, CASP6, CASP7, CASP8, CASP9, CFLAR (CASPER), CRADD, PYCARD (TMS1/ASC), ABL1, AKT1, BAD, BAK1, BAX, BCL2L11, BCLAF1, BID, BIK, BNIP3, BNIP3L, CASP1 (ICE), CASP10 (MCH4), CASP14, CASP2, CASP4, CASP6, CASP8, CD70 (TNFSF7), CIDEB, CRADD, FADD, FASLG (TNFSF6), HRK, LTA (TNFB), NOD1 (CARD4), PYCARD (TMS1/ASC), RIPK2, TNF, TNFRSF10A, TNFRSF10B (DR5), TNFRSF25 (DR3), TNFRSF9, TNFSF10 (TRAIL), TNFSF8, TP53, TP53BP2, TRADD, TRAF2, TRAF3, TRAF4, and a combination thereof. In some embodiments, conditions to suppress expression of apoptosis-associated genes comprise administration of one or more antisense oligonucleotides. The antisense oligonucleotide can activate RNAse H. In some embodiments, conditions to suppress expression of apoptosis-associated genes comprise administration of one or more agents capable of inducing RNA interference. The agent can comprise short interfering RNA and/or short hairpin RNA.

In some embodiments of the methods and systems, the fibroblast cells are administered locally into an area of degeneration or are administered systemically. An area of degeneration can comprise atrophy or loss of function in cells or tissues. In some embodiments, the fibroblast cells are administered in a formulation with a volume of between about 0.1 ml and about 200 ml. In some embodiments, one or more additional therapeutic agents are administered in combination with fibroblast cells locally or systemically. The one or more additional therapeutic agents can be selected from the group consisting of one or more growth factors, one or more differentiation factors, regenerative cells, one or more nutritional supplements, and a combination thereof. In some embodiments, the one or more additional therapeutic agents are a growth factor. In some embodiments, the one or more additional therapeutic agents are stromal derived factor 1. In some embodiments, the one or more therapeutic agents comprise platelet concentrate. The one or more additional therapeutic agents and the fibroblast cells can be administered using a pharmaceutically acceptable carrier. In some embodiments, the pharmaceutically acceptable carrier is selected from the group consisting of beads, microspheres, nanospheres, hydrogels, gels, polymers, ceramics, collagen, platelet gels, and a combination thereof. In some embodiments, the carrier comprises a hydrogel. In some embodiments, the carrier comprises microspheres. In some embodiments, the one or more additional therapeutic agents are administered simultaneously with administration of fibroblast cells. In some embodiments, the one or more additional therapeutic agents are administered prior to administration of fibroblast cells. In some embodiments, the one or more additional therapeutic agents are administered after administration of fibroblast cells.

In some embodiments of the methods and systems, the fibroblast cells are treated with one or more factors capable of stimulating smooth muscle differentiation. The factors capable of stimulating smooth muscle differentiation can be selected from the group consisting of IL-10, IL-20, IL-25, GDF-5, GDF-11, BMP-13, MIA/CD-RAP, PDGF-BB, FGF, IGF, dexamethasone, and a combination thereof.

In some embodiments, ESWT of the methods or systems is administered by a shockwave generating device in addition to, including together with. the fibroblast cells. The shockwave generating device can be utilized in an aqueous environment. The ESWT regimen or system can be administered to a focal zone in cells or tissue. The focal zone can comprise reduced circulation, degenerative features, or a combination thereof, and degenerative features can comprise atrophy or loss of function.

In some embodiments, the ESWT regimen or system promotes at least one or more of the following biological activities in the focal zone: (a) inducing of angiogenesis; (b) modulating the immune system; (c) suppressing inflammation; (d) preventing of tissue atrophy; (e) regenerating of functional tissue; (f) inhibition of neuronal cell dysfunction; and (g) inhibition of smooth muscle degeneration. The ESWT regimen or system can comprise a treatment regimen selected based on at least one parameter selected from the group consisting of waveform parameters, treatment protocol parameters, anatomical parameters, and a combination thereof. The waveform parameters can comprise wave number, frequency, and intensity. In some embodiments, the wave intensity is about from about 50 bar to about 200 bar. In some embodiments, the wave frequency is from about 60 to about 300 shockwaves per minute. In some embodiments, the wave number is up to about 3500 per ESWT session. In some embodiments, the anatomical parameters comprise at least one focal zone to be treated. In some embodiments, the at least one focal zone comprises up to about 90% of one or more areas identified as being subject to degenerative changes in cells or tissue. The degenerative changes can comprise atrophy or loss of function in cells or tissue. In some embodiments, the ESWT regimen or system is combined with a drug, cellular treatment, or a combination thereof. The cellular treatment can comprise mesenchymal stem cells, hematopoietic stem cells, or embryonic-like stem cells.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description. It is to be expressly understood, however, that specific embodiments are provided for the purpose of illustration and description only and are not intended as a definition of the limits of the present disclosure.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

“Fibroblasts” refer to a cell, progenitor cell, or differentiated cell and include isolated fibroblast cells or population(s) thereof capable of proliferating and differentiating into ectoderm, mesoderm, or endoderm. As used herein, placental and adult-derived cellular populations are included in the definition of “fibroblasts.” In the context of the present disclosure, fibroblast cells may be derived from sources including at least cord blood, placenta, bone marrow, amnion, intraventricular cells from the cerebral spinal fluid, circulating fibroblast cells, mesenchymal stem cell associated cells, germinal cells, adipose tissue, exfoliated tooth derived fibroblasts, hair follicle, dermis, parthenogenically-derived fibroblasts, fibroblasts that have been reprogrammed to a dedifferentiated state, side population-derived fibroblasts, and the like.

As used herein, “cell culture” means conditions wherein cells are obtained (e.g., from an organism) and grown under controlled conditions (“cultured” or grown “in culture”) outside of an organism. A primary cell culture is a culture of cells taken directly from an organism (e.g., tissue cells, blood cells, cancer cells, neuronal cells, fibroblasts, etc.). Cells are expanded in culture when placed in a growth medium under conditions that facilitate cell growth and/or division. The term “growth medium” means a medium sufficient for culturing cells. Various growth media may be used for the purposes of the present disclosure including, for example, Dulbecco's Modified Eagle Media (also known as Dulbecco's Minimal Essential Media) (DMEM), or DMEM-low glucose (also DMEM-LG herein). DMEM-low glucose may be supplemented with fetal bovine serum (e.g., about 10% v/v, about 15% v/v, about 20% v/v, etc), antibiotics, antimycotics (e.g., penicillin, streptomycin, and/or amphotericin B), and/or 2-mercaptoethanol. Other growth media and supplementations to growth media are capable of being varied by the skilled artisan. The term “standard growth conditions” refers to culturing cells at 37° C. in a standard humidified atmosphere comprising 5% CO₂. While such conditions are useful for culturing, it is to be understood that such conditions are capable of being varied by the skilled artisan who will appreciate the options available in the art for culturing cells. When cells are expanded in culture, the rate of cell proliferation is sometimes measured by the amount of time needed for the cells to double in number. This is referred to as doubling time.

“Differentiation” (e.g., cell differentiation) describes a process by which an unspecialized (or “uncommitted”) or less specialized cell acquires the features (e.g., gene expression, cell morphology, etc.) of a specialized cell, such as a nerve cell or a muscle cell for example. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. In some embodiments of the disclosure, “differentiation” of fibroblasts to neuronal cells is described. This process may also be referred to as “transdifferentiation”.

As used herein, “dedifferentiation” refers to the process by which a cell reverts to a less specialized (or less committed) position within the lineage of a cell. As used herein, the lineage of a cell defines the heredity of the cell. The lineage of a cell places the cell within a hereditary scheme of development and differentiation. Within the context of the current disclosure, “dedifferentiation” may refer to fibroblasts acquiring more “immature” associated markers such as OCT4, NANOG, CTCFL, ras, raf, SIRT2, and SOX2. Additionally, “dedifferentiation” may mean acquisition of functional properties such as enhanced proliferation activity and/or migration activity towards a chemotactic gradient. In some embodiments fibroblasts may be “dedifferentiated” by treatment with various conditions, subsequent to which they are “differentiated” into other cell types.

As used herein, the term “therapeutically effective amount” is synonymous with “effective amount”, “therapeutically effective dose”, and/or “effective dose” refers to an amount of an agent sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. In one embodiment, the term therapeutically effective amount and like terms are used to refer to the frequencies and concentrations at which fibroblast cells are administered for treating a degenerative condition. An effective amount of cells may be between 10³ and 10¹¹ cells. In some cases, an effective amount of cells is about 10⁴ cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

As used herein, “therapeutic energy treatment” refers to any light-, acoustic-, electrical-, or mechanical means of stimulating cells or tissue through means applied either externally to cells (i.e. regenerative cells) or directly to the tissue for therapeutic purposes.

As used herein, “extracorporeal shockwave” refers to a continuous single sound wave generated by a specific sound generator, which has a high peak pressure amplitude of up to 100 MPa, has a short duration of less than 1 μs, and delivers to a specific target area with an energy density in the range of 0.005 mJ/mm² to 1.0 mJ/mm².

As used herein, “biological dysfunction” means loss or inhibition of the normal functioning of a tissue or of cells. In the context of cardiac dysfunction, reduction in stroke volume or ejection fraction as compared to normal is defined as a dysfunction. In some embodiments, suppression of immunity is a biological dysfunction.

As used herein, “preactivation” means induction of biochemical processes within the graft so as to allow for increased viability, augmented function, accelerated integration with the tissue in which implantation of cellular graft has occurred, or a combination thereof. The use of extracorporeal ultrasound is disclosed for preactivating fibroblasts cells, particularly fibroblast cells possessing regenerative activity. Also disclosed is a means of “preactivating” a cellular graft before implantation. One embodiment encompasses preactivation as a means of augmenting recovery from a chronic condition; in further embodiments, tissue regeneration is induced using modified fibroblasts alone or together with treatment by extracorporeal ultrasound shock waves.

As used herein, “regenerative activities” include but are not limited to therapeutic functions, stimulation of angiogenesis, inhibition of inflammation, and/or augmentation of tissue self-renewal, for example in part through activation of endogenous and/or exogenous stem and/or progenitor cells. In one embodiment, extracorporeal shock waves are used to augment the regenerative activities of fibroblasts. Regenerative activities include the promotion of angiogenesis, suppression of inflammation, and secretion of growth factors such as IGF-1, EGF-1, FGF-2, VEGF, and/or FGF-11. In some embodiments, regenerative activity is mediated by stimulating recruitment of stem cells from various anatomical niches such as the bone marrow into systemic circulation. In a further embodiment, fibroblasts having regenerative activities are isolated for specific markers and subsequently transfected with genes capable of endowing various therapeutic functions. Genes useful for stimulation of regenerative activities including augmentation of hematopoietic activity include interleukin-12 and interleukin-23 to stimulate proliferation of hematopoietic stem cells, for example. Other useful genes include interleukin-35, wherein interleukin-35 transfection allows for generation of cells possessing anti-inflammatory and angiogenic T regulatory cell activity, said cells possessing T regulatory cell activities include cells expressing the transcription factor FoxP3.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition. For subjects having or suspected of having a medical condition, the medical condition may be of one or more types. The subject may have a disease or be suspected of having the disease. The subject may or may not be asymptomatic. The subject may be of any gender. The subject may be an infant, child, adolescent, or adult. The subject may be of a certain age, such as less than 1 years of age or at least 1, 5, 10, 15, 20, 25, 30, 35, 40, 45, 50, 55, 60, 65, 70, 75, 80, 85, 90, 95, or 100 or more years of age.

### II. Generation of Regenerative Fibroblasts

Certain aspects of the present disclosure relate to the generation of regenerative fibroblast cells for treatment of individuals alone or in combination with ESWT. Regenerative fibroblast cells may be generated by culturing fibroblasts under sufficient conditions to generate a regenerative fibroblast cell. In some embodiments, the fibroblast cells can provide a tissue with regenerative activity. In some embodiments, the method includes culturing the population of fibroblast regenerative cells under conditions that support proliferation of the cells. In additional embodiments, the fibroblast cells may be cultured under conditions that form tissue aggregate bodies. In some embodiments, the fibroblast cells are used to create other cell types for tissue repair or regeneration. Generation of fibroblasts has been described previously in the art and is incorporated herein. For example, fibroblasts can be extracted from dermal biopsy samples by means of collagenase digestion or mechanical separation. Fibroblasts can then expanded in tissue culture. In some embodiments, specific subsets of fibroblasts are extracted and propagated. For example, in one embodiment, selection of CD73-expressing fibroblasts is performed using magnetic bead separation.

Specific desirable properties of fibroblast cells of the present disclosure are the ability to increase endothelial function; induce neoangiogenesis; prevent atrophy; differentiate into functional tissue; induce local resident stem and/or progenitor cells to proliferate through secretion of soluble factors or membrane bound activities; and/or induce immune modulation by suppressing expression of inflammatory genes in immune cells. For example, in one embodiment, suppression of IL-17 expression by monocytes is mediated by fibroblasts of the disclosure. In one embodiment, fibroblast cells are collected from an autologous patient, expanded ex vivo, and reintroduced into the patient at a concentration and frequency sufficient to cause therapeutic benefit. The fibroblast cells are selected for the ability to cause neoangiogenesis, prevent tissue atrophy, and regenerate functional tissue. In another embodiment, fibroblasts are utilized from allogeneic or xenogeneic sources.

When selecting fibroblast cells, several factors must be taken into consideration, including the ability for ex vivo expansion without loss of therapeutic activity, ease of extraction, general potency of activity, and potential for adverse effects. Ex vivo expansion ability of fibroblasts can be measured using typical proliferation and colony assays known to one skilled in the art, while identification of therapeutic activity depends on functional assays that test biological activities correlated with therapeutic goals.

In some embodiments, assessment of therapeutic or regenerative activity is performed using surrogate assays which detect one or more markers associated with a specific therapeutic activity. In some embodiments, assays used to identify therapeutic activity of fibroblast cell populations include evaluation of the production of one or more factors associated with desired therapeutic activity. In some embodiments, evaluation of the production of one or more factors to approximate therapeutic activity in vivo includes identification and quantification of the production of FGF, VEGF, angiopoietin, HGF, IGF, PDGF, interleukin 1 receptor antagonist, interleukin 10, and HLA-G, a combination thereof, or other angiogenic molecules that may be used to serve as a guide for approximating therapeutic activity in vivo.

In some embodiments, the regenerative fibroblast cells used in combination with ESWT are capable of proliferating and differentiating into ectoderm, mesoderm, or endoderm. In some embodiments, the enriched population of fibroblast cells are about 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 micrometers in size. In some embodiments, the fibroblast cells are expanded in culture using one or more cytokines, one or more chemokines and/or one or more growth factors prior to administration to an individual in need thereof. The agent capable of inducing fibroblast expansion can be selected from the group consisting of TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LW, PDGF, a member of the bone morphogenic protein family (BMP1, BMP2, BMP3, BMP4, BMP5, BMP6, BMP7, BMP8a, BMP8b, BMP10, BMP11, BMP15), and a combination thereof. The agent capable of inducing fibroblast differentiation can be selected from the group consisting of HGF, BDNF, VEGF, FGF1, FGF2, FGF4, FGF20, and a combination thereof.

Conditions promoting certain types of fibroblast proliferation or differentiation can be used during the culture of regenerative fibroblast cells. These conditions include but are not limited to alteration in temperature, oxygen/carbon dioxide content, and/or turbidity of the growth media, and/or exposure to small molecule modifiers of cell culture-like nutrients, certain enzyme inhibitors, certain enzyme stimulators, and/or histone deacetylase inhibitors, such as valproic acid. In some embodiments, regenerative fibroblast cells may be administered to an individual in need thereof via one or more of intravenous, intramuscular, intraperiotoneal, transdermal, by infusion, and/or parenteral administration, for example.

In some embodiments, fibroblast cells are cultured under conditions to suppress expression of one or more apoptosis-associated genes. Anti-apoptosis genes allow for enhanced survival of fibroblasts in vitro and in vivo. In some embodiments, the one or more apoptosis-associated genes are selected from the group consisting of Fas, FasL, CASP1 (ICE), CASP10 (MCH4), CASP14, CASP2, CASP3, CASP4, CASP5, CASP6, CASP7, CASP8, CASP9, CFLAR (CASPER), CRADD, PYCARD (TMS1/ASC), ABL1, AKT1, BAD, BAK1, BAX, BCL2L11, BCLAF1, BID, BIK, BNIP3, BNIP3L, CASP1 (ICE), CASP10 (MCH4), CASP14, CASP2, CASP4, CASP6, CASP8, CD70 (TNFSF7), CIDEB, CRADD, FADD, FASLG (TNFSF6), HRK, LTA (TNFB), NOD1 (CARD4), PYCARD (TMS1/ASC), RIPK2, TNF, TNFRSF10A, TNFRSF10B (DR5), TNFRSF25 (DR3), TNFRSF9, TNFSF10 (TRAIL), TNFSF8, TP53, TP53BP2, TRADD, TRAF2, TRAF3, TRAF4, and a combination thereof.

In some embodiments, the conditions to suppress expression of apoptosis-associated genes comprise administration of an antisense oligonucleotide which activates RNAse H. In some embodiments, the conditions to suppress expression of apoptosis-associated genes comprise administration of an agent capable of inducing RNA interference, including short interfering RNA and/or short hairpin RNA. The cells may be modified by CRISPR to reduce expression of one or more apoptosis-associated genes.

In some embodiments, fibroblasts are cultured under hypoxic conditions prior to administration in order to confer enhanced cytokine production properties and stimulate migration toward chemotactic gradients. Without wishing to be bound theory, protocols to enhance the regenerative potential of non-fibroblast cells using hypoxia can be modified or adapted for use with fibroblasts. For example, in one study, short-term exposure of MSCs to 1% oxygen increased mRNA and protein expression of the chemokine receptors CX3CR1 and CXCR4. After 1-day exposure to low oxygen, in vitro migration of MSCs in response to the fractalkine and SDF-1alpha increased in a dose dependent manner, while blocking antibodies for the chemokine receptors significantly decreased migration. Xenotypic grafting of cells from hypoxic cultures into early chick embryos demonstrated more efficient grafting of cells from hypoxic cultures compared to cells from normoxic cultures, and cells from hypoxic cultures generated a variety of cell types in host tissues. Other descriptions of hypoxic conditioning are described in the art. For example, cells can be cultured in hypoxic conditions or with gases that displace oxygen and/or cells can be treated with hypoxic mimetics.

In some embodiments chemical agents such as iron chelators, for example, deferoxamine, are added during in vitro incubation or in vivo to enhance migration of fibroblasts to an area in need. Another useful preconditioning agent is all trans retinoic acid, used at concentrations similar to those described for MSC, for example, between 0.001 uM to 1 uM or between 0.01 uM and 1 uM.

Without wishing to be bound by theory, hypoxia has been demonstrated to induce expression of angiogenic genes in cells. For example, studies involving hypoxic preconditioning (HPC) of MSC exposed MSCs to 0.5% oxygen for 24, 48, or 72 h before evaluating the expression of prosurvival, proangiogenic, and functional markers, such as hypoxia-inducible factor-1α, VEGF, phosphorylated Akt, survivin, p21, cytochrome c, caspase-3, caspase-7, CXCR4, and c-Met. MSCs exposed to 24-h hypoxia showed reduced apoptosis and had significantly higher levels of prosurvival, proangiogenic, and prodifferentiation proteins compared to MSCs exposed to 72-h hypoxia. Cells taken directly from a cryopreserved state did not respond as effectively to 24-h HPC as those cells cultured under normoxia before HPC. Cells cultured under normoxia before HPC showed decreased apoptosis and enhanced expression of connexin-43, cardiac myosin heavy chain, and CD31. The preconditioned cells were also able to differentiate into cardiovascular lineages. The results of the study suggest that MSCs cultured under normoxia before 24-h HPC are in a state of optimal expression of prosurvival, proangiogenic, and functional proteins that may increase subsequent survival after engraftment of the cells.

Thus, in some embodiments, regenerative fibroblast cells are exposed to 0.1% to 10% oxygen for a period of 30 minutes to 3 days. In some embodiments, regenerative fibroblast cells are exposed to 3% oxygen for 24 hours. In some embodiments, regenerative fibroblast cells are exposed to cobalt (II) chloride to chemically induce hypoxia. In some embodiments, regenerative fibroblast cells are exposed to cobalt (II) chloride for 1 to 48 hours. In some embodiments, regenerative fibroblast cells are exposed to cobalt (II) chloride for 24 hours. In some embodiments, regenerative fibroblast cells are exposed to 1 μM-300 μM. In some embodiments, regenerative fibroblast cells are exposed to 250 μM cobalt (II) chloride. In some embodiments, hypoxia induces an upregulation in HIF-1α, which is detected by expression of VEGF secretion. In some embodiments, hypoxia induces an upregulation of CXCR4 on fibroblast cells, which promotes homing of the cells to an SDF-1 gradient in inflamed areas.

In some embodiments, the fibroblast cells express proteins characteristic of normal fibroblasts including the fibroblast-specific marker, CD90 (Thy-1), a 35 kDa cell-surface glycoprotein, and/or the extracellular matrix protein, collagen. In some embodiments, fibroblast cells express at least one of Oct-4, Nanog, Sox-2, KLF4, c-Myc, Rex-1, GDF-3, LIF receptor, CD105, CD117, CD344 or Stella markers. In some embodiments, fibroblast cells do not express at least one of MHC class I, MHC class II, CD45, CD13, CD49c, CD66b, CD73, CD105, or CD90 cell surface proteins. In some embodiments, the method optionally includes the step of depleting cells expressing stem cell surface markers or MHC proteins from the cell population, thereby isolating a population of stem cells. In some embodiments, the cells to be depleted express MHC class I, CD66b, glycophorin a, or glycophorin b. In some embodiments, fibroblast cells are isolated and expanded and possess one or more markers selected from the group consisting of CD10, CD13, CD44, CD73, CD90, CD141, PDGFr-alpha, HLA-A, HLA-B, HLA-C, and a combination thereof. In some embodiments, the fibroblast cells do not produce one or more of CD31, CD34, CD45, CD117, CD141, HLA-DR, HLA-DP, HLA-DQ, or a combination thereof. In further embodiments, the fibroblast regenerative cell has enhanced expression of GDF-11 as compared to a control. In still further embodiments, the fibroblast cells express CD73, which is indicative of fibroblast cells having regenerative activity.

Fibroblast cells used in the disclosed methods can undergo at least 25, 30, 35, or 40 doublings prior to reaching a senescent state. Methods for deriving cells capable of doubling to reach 10¹⁴ cells or more are provided. In some embodiments, methods are used to derive cells that can double sufficiently to produce at least about 10¹⁴, 10¹⁵, 10¹⁶, or 10¹⁷ or more cells when seeded at from about 10³ to about 10⁶ cells/cm² in culture within 80, 70, or 60 days or less. In some embodiments, fibroblasts are transfected with genes to allow for enhanced growth and overcoming of the Hayflick limit.

In some embodiments, the method optionally includes enriching populations of fibroblast cells. In one embodiment, cells are transfected with a polynucleotide vector containing a stem cell-specific promoter operably linked to a reporter or selection gene. In some embodiments, the cell-specific promoter is an Oct-4, Nanog, Sox-9, GDF3, Rex-1, or Sox-2 promoter. In some embodiments, the method further includes the step of enriching the population for the regenerative fibroblast cells using expression of a reporter or selection gene. In some embodiments, the method further includes the step of enriching the population of the regenerative fibroblast cells by flow cytometry. In a further embodiment, the method further comprises the steps of selecting fibroblast cells expressing CD105 and/or CD 117 and transfecting the fibroblast cells expressing CD105 and/or CD117 with cell-permanent NANOG gene.

In another embodiment, the method further includes the steps of contacting the fibroblast cells with a detectable compound that enters the cells, the compound being selectively detectable in proliferating and non-proliferating cells and enriching the population of cells for the proliferating cells. In some embodiments, the detectable compound is carboxyfluorescein diacetate, succinimidyl ester, and/or Aldefluor.

In some embodiments, fibroblast regenerative cells comprise fibroblast side population cells isolated based on expression of the multidrug resistance transport protein (ABCG2) or the ability to efflux intracellular dyes such as rhodamine-123 and or Hoechst 33342. Without being bound to theory, cells possessing this property express stem-like genes and are known for enhanced regenerative ability compared to cells which do not possess this efflux mechanism. Fibroblast side population cells may be derived from tissues including pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, or a combination thereof.

In some embodiments, the fibroblast dosage formulation is a suspension of fibroblasts obtained from a biopsy using standard tissue culture procedures, and the donor providing the biopsy may be either the individual to be treated (autologous), or the donor may be different from the individual to be treated (allogeneic). In some embodiments, the fibroblast cells are xenogenic. In some embodiments wherein allogeneic fibroblast cells are utilized for an individual, the fibroblast cells come from one or a plurality of donors. In some embodiments fibroblasts are used from young donors. In some embodiments, steps are taken to protect allogeneic or xenogenic cells from immune-mediated rejection by the recipient. Steps include encapsulation, co-administration of an immune suppressive agent, transfection of the cells with one or more immune suppressory agents, or a combination thereof. In some embodiments, tolerance to the cells is induced through immunological means.

In one embodiment, biopsies are from skin tissue (dermis and epidermis layers) from a subject's post-auricular area. In one embodiment, the starting material is composed of three 3-mm punch skin biopsies collected using standard aseptic practices. The biopsies are collected by the treating physician, placed into a vial containing sterile phosphate buffered saline (PBS), and stored at 2-8° C. Upon initiation of the process, biopsies are inspected, and accepted biopsy tissues are washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Other commercially available collagenases may also be used, such as Serva Collagenase NB6 (Helidelburg, Germany).

After digestion, Iscove's Complete Growth Media (IMDM, GA, 10% Fetal Bovine Serum (FBS)) is added to neutralize the enzyme, and cells are pelleted by centrifugation and resuspended in 5.0 mL IMDM. Alternatively, full enzymatic inactivation is achieved by adding IMDM without centrifugation. Additional IMDM is added prior to seeding of the cell suspension into a particular flask (such as a T-175 cell culture flask) for initiation of cell growth and expansion. Alternatively, a T-75, T-150, T-185, or T-225 flask can be used in place of the T-175 flask. Cells are incubated at 37.0±2° C. with 5.0±1.0% CO₂ and supplemented with fresh IMDM every three to five days by removing half of the IMDM and replacing it with the same volume with fresh media. Alternatively, full IMDM replacements can be performed.

In specific cases, cells are not cultured in the T-175 flask for more than 30 days prior to passaging. Confluence is monitored throughout the process to ensure adequate seeding densities upon culture splitting. When cell confluence is greater than or equal to 40% in the T-175 flask, the cells are passaged by removing the spent media, washing the cells, and treating the cells with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF), or a Two Layer Cell Stack (2 CS) can be used in place of the T-500 Flask.

Morphology may be evaluated at each passage and prior to harvest to monitor culture purity throughout the process by comparing the observed sample with visual standards for morphological examination of cell cultures. Typical fibroblast morphologies when growing in cultured monolayers include elongated, fusiform, or spindle-shaped cells with slender extensions or larger, flattened stellate cells with cytoplasmic leading edges. A mixture of these morphologies may also be observed. Fibroblasts in less confluent areas can be similarly shaped but randomly oriented. The presence of keratinocytes in cell cultures may also be evaluated. Keratinocytes are round and irregularly shaped and, at higher confluence, appear to be organized in a cobblestone formation. At lower confluence, keratinocytes are observable in small colonies.

Cells are incubated at 37±2° C. with 5.0±1.0% CO₂ and passaged every three to five days for cells in T-500 flasks and every five to seven days for cells in ten layer cell stacks (10 CS). Cells should not be cultured for more than 10 days prior to passaging. When cell confluence in a T-500 flask is ≥95%, cells are passaged to a 10 Layer Cell Stack (10 CS) culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to the 10 CS. Additional IMDM is added to neutralize the trypsin, and the cells from the T-500 flask are pipetted into a 2 L bottle containing fresh IMDM.

The contents of the 2 L bottle may be transferred into the 10 CS and seeded across all layers of the 10 CS. Cells are then incubated at 37.0±2° C. with 5.0±1.0% CO₂ and supplemented with fresh IMDM every five to seven days. Cells should not be cultured in the 10 CS for more than 20 days prior to passaging. In one embodiment, the passaged fibroblasts are rendered substantially free of immunogenic proteins present in the culture medium by incubating the expanded fibroblasts for a period of time in protein-free medium. When cell confluence in the 10 CS is ≥95%, cells are harvested. Harvesting is performed by removing the spent media, washing the cells, treating with Trypsin-EDTA to release adherent cells into the solution, and adding additional IMDM to neutralize the trypsin. Cells are collected by centrifugation and resuspended, and in-process QC testing is performed to determine total viable cell count and cell viability as well as sterility and the presence of endotoxins.

In one embodiment, regenerative fibroblast cells are purified from cord blood. Cord blood fibroblast cells are fractionated, and the fraction with enhanced therapeutic activity is administered to the patient. In some embodiments, cells with therapeutic activity are enriched based on physical differences (e.g., size and weight), electrical potential differences (e.g., charge on the membrane), differences in uptake or excretion of certain compounds (e.g., rhodamine-123 efflux), as well as differences in expression marker proteins (e.g., CD73). Distinct physical property differences between stem cells with high proliferative potential and low proliferative potential are known. Accordingly, in some embodiments, cord blood fibroblast cells with a higher proliferative ability are selected, whereas in other embodiments, a lower proliferative ability is desired. In some embodiments, cells are directly injected into the area of need and should be substantially differentiated. In other embodiments, cells are administered systemically and should be less differentiated, so as to still possess homing activity to the area of need.

In embodiments where specific cellular physical properties are the basis of differentiating between cord blood fibroblast cells with various biological activities, discrimination on the basis of physical properties can be performed using a Fluorescent Activated Cell Sorter (FACS), through manipulation of the forward scatter and side scatter settings. Other embodiments include methods of separating cells based on physical properties using filters with specific size ranges, density gradients, and pheresis techniques. In embodiments where differentiation is based on electrical properties of cells, techniques such as electrophotoluminescence are used in combination with a cell sorting means such as FACS. In some embodiments, selection of cells is based on ability to uptake certain compounds as measured by the ALDESORT system, which provides a fluorescent-based means of purifying cells with high aldehyde dehydrogenase activity. Without being bound by theory, cells with high levels of this enzyme are known to possess higher proliferative and self-renewal activities in comparison to cells possessing lower levels. Further embodiments include methods of identifying cells with high proliferative activity by identifying cells with ability to selectively efflux certain dyes such as rhodamine-123, Hoechst 33342, or a combination thereof. Without being bound to theory, cells possessing this property often express the multidrug resistance transport protein ABCG2 and are known for enhanced regenerative ability compared to cells which do not possess this efflux mechanism.

In some embodiments, cord blood cells are purified for certain therapeutic properties based on the expression of markers. In one particular embodiment, cord blood fibroblast are purified for cells with the endothelial precursor cell phenotype. Endothelial precursor cells or progenitor cells express markers such as CD133, CD34, or a combination thereof and are purified by positive or negative selection using techniques such as magnetic activated cell sorting (MACS), affinity columns, FACS, panning, other means known in the art, or a combination thereof. In some embodiments, cord blood-derived endothelial progenitor cells are administered directly into the target tissue, while in other embodiments, the cells are administered systemically. In some embodiments, the endothelial precursor cells are differentiated in vitro and infused into a patient. Verification of endothelial differentiation is performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

In some embodiments, cord blood fibroblast cells are endowed with desired activities prior to administration into the patient. In one specific embodiment, cord blood cells are “activated” ex vivo by brief culture in hypoxic conditions to upregulate nuclear translocation of the HIF-1α transcription factor and endow the cord blood cells with enhanced angiogenic potential. In some embodiments, hypoxia is achieved by culture of cells in conditions of 0.1% oxygen to 10% oxygen. In further embodiments, hypoxia is achieved by culture of cells in conditions of 0.5% oxygen and 5% oxygen. In further embodiments, hypoxia is achieved by culture of cells in conditions of about 1% oxygen. Cells may be cultured for a variety of time points ranging from 1 hour to 72 hours in some embodiments, to 13 hours to 59 hours in further embodiments and around 48 hours in still further embodiments. In one embodiment, cord blood cells are assessed for angiogenic or other desired activities prior to administration of the cord blood cells into the patient. Assessment methods are known in the art and include measurement of angiogenic factors, the ability to support cell viability and activity, and the ability to induce regeneration of the cellular components.

In additional embodiments, cord blood fibroblast cells are endowed with additional therapeutic properties through treatment ex vivo with factors such as de-differentiating compounds, proliferation-inducing compounds, compounds known to endow and/or enhance cord blood cells with useful properties, or a combination thereof. In one embodiment, cord blood cells are cultured with an inhibitor of the enzyme GSK-3 to enhance expansion of cells with pluripotent characteristics while maintaining the rate of differentiation. In another embodiment, cord blood cells are cultured in the presence of a DNA methyltransferase inhibitor such as 5-azacytidine to confer a “de-differentiation” effect. In another embodiment cord blood fibroblast cells are cultured in the presence of a differentiation agent that induces the cord blood stem cells to generate enhanced numbers of cells useful for treatment after the cord blood cells are administered to a patient.

In one embodiment, regenerative fibroblasts are purified from placental tissues. In contrast to cord blood fibroblast cells, in some embodiments, placental fibroblast cells are purified directly from placental tissues including the chorion, amnion, and villous stroma. In another embodiment, placental tissue is mechanically degraded in a sterile manner and treated with enzymes to allow dissociation of the cells from the extracellular matrix. Such enzymes include but are not restricted to trypsin, chymotrypsin, collagenases, elastase, hyaluronidase, or a combination thereof. In some embodiments, placental cell suspensions are subsequently washed, assessed for viability, and used directly by administration locally or systemically. In some embodiments, placental cell suspensions are purified to obtain certain populations with increased biological activity.

Purification may be performed using means known in the art including those used for purification of cord blood fibroblast cells. In some embodiments, purification may be achieved by positive selection for cell markers including SSEA3, SSEA4, TRA1-60, TRA1-81, c-kit, and Thy-1. In some embodiments, cells are expanded before introduction into the human body. Expansion can be performed by culture ex vivo with specific growth factors. Embodiments described for cord blood and embryonic stem also apply to placental stem cells.

In some embodiments, fibroblasts are obtained from a source selected from the group consisting of dermal fibroblasts; placental fibroblasts; adipose fibroblasts; bone marrow fibroblasts; foreskin fibroblasts; umbilical cord fibroblasts; amniotic fluid; embryonic fibroblasts; hair follicle-derived fibroblasts; nail-derived fibroblasts; endometrial-derived fibroblasts; keloid-derived fibroblasts; ear lobe skin; plastic surgery-related by-products; and a combination thereof.

In some embodiments, fibroblasts are fibroblasts isolated from placenta, umbilical cord, cord blood, peripheral blood, omentum, hair follicle, skin, bone marrow, adipose tissue, or Wharton's Jelly. In some embodiments, the fibroblasts are fibroblasts isolated from peripheral blood of a subject who has been exposed to conditions sufficient to stimulate fibroblasts from the subject to enter the peripheral blood. In another embodiment, fibroblast cells are mobilized by use of a mobilizing agent or therapy for treatment in conjunction with ESWT. In some embodiments, the conditions and/or agents sufficient to stimulate fibroblasts from the subject to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof. In some embodiments, the mobilization therapy is selected from a group comprising exercise, hyperbaric oxygen, autohemotherapy by ex vivo ozonation of peripheral blood, induction of SDF-1 secretion in an anatomical area outside of the bone marrow, or a combination thereof. In some embodiments, the committed fibroblasts can express the marker CD133 or CD34 and are mobilized. In some embodiments, extracorporeal shock wave ultrasound may be used to direct, or concentrate, mobilized fibroblasts to a particular tissue.

### III. Differentiation of Fibroblasts into Neuronal Cells

In some embodiments, fibroblasts are induced to differentiate into cells possessing properties of neurons cells, or neuron cells themselves. In some embodiments, one or more agents that act as “regenerative adjuvants” are administered to fibroblasts to generate neurons. In some embodiments, neuron-like cells prepared from fibroblasts are exposed to ESWT, which improves the longevity of the neuron-like cells. In some embodiments, the ESWT-treated neurons are used therapeutically to treat neuronal cell dysfunction of any kind.

According to some embodiments, fibroblasts are incubated with one or more growth factors (i.e., mitogenic compounds) under suitable growth conditions to allow for cell proliferation and to prepare the cells for differentiation into neuronal cells. In some embodiments, the fibroblasts are incubated with one or more differentiation inducers or inducing agents, and optionally, one or more growth factors under conditions suitable to allow for differentiation and/or propagation of a variety of cell types. As one of ordinary skill would recognize, several known compounds function as both growth factors and differentiation inducers. Growth factors include but are not limited to M-CSF, IL-6, LIF, and IL-12. Examples of compounds functioning as growth factors and/or differentiation inducers include but are not limited to lipopolysaccharide (LPS), phorbol 12-myristate 13-acetate (PMA), stem cell growth factor, human recombinant interleukin-2 (IL-2), interleukin-3 (IL-3), epidermal growth factor (EGF), b-nerve growth factor (bNGF), recombinant human vascular endothelial growth factor165 isoform (VEGF₁₆₅), and/or hepatocyte growth factor (HGF). In various embodiments, doses of growth factors and/or differentiation inducers used to promote fibroblast proliferation and/or increase susceptibility of fibroblasts to differentiation include but are not limited to: 0.5 ng/ml to 1.0 μg/ml LPS, 1 to 160 nM PMA, 500-2400 units/ml bNGF, 12.5-100 ng/ml VEGF, 10-200 ng/ml EGF, and 25-200 ng/ml HGF.

Induction of fibroblast differentiation to neurons may be performed using several means. In some embodiments, protocols useful for differentiating mesenchymal stem cells into neurons may be adapted, modified, or replicated using fibroblasts as the starting population instead of MSCs. Such protocols are known in the art and incorporated by reference. For example, fibroblasts may be treated with agents such as BDNF, NGF, or CNTF in vitro for a period of 1 hour to 2 weeks (including 1 hr to 1 week, 1 hr to 6, 5, 4, 3, 2, or 1 days, or any range derivable therein) to induce generation of neurons or neuronal like cells. In some embodiments, fibroblasts are co-cultured with media conditioned by neurons, which is sufficient to induce some degree of fibroblasts differentiation. In further embodiments, “dedifferentiating” agents such as valproic acid are used in combination with neuron-conditioned media to enhance the differentiation of neurons from fibroblasts. Valproic acid may also be used to enhance differentiation of other cells from fibroblasts, including with a specific media, in some cases.

In some embodiments, fibroblasts are first treated with one or more dedifferentiating agents, such as one or more of valproic acid, lithium, 5-azacytidine, or a combination thereof, to induce expression of one or more markers, including OCT-4, alkaline phosphatase, Sox2, TDGF-1, SSEA-3, SSEA-4, TRA-1-60, TRA-1-80, or a combination thereof. Prior to neuronal formation, dedifferentiated fibroblasts cells may be cultured on a matrix. In some embodiments, the matrix is selected from the group consisting of laminin, fibronectin, vitronectin, proteoglycan, entactin, collagen, collagen I, collagen IV, collagen VIII, heparan sulfate, MATRIGEL™ (a soluble preparation from Engelbreth-Holm-Swarm (EHS) mouse sarcoma cells), CELLSTART™, a human basement membrane extract, and any combination thereof.

Dedifferentiated fibroblasts may be cultured in a multilayer population or embryoid body for a time sufficient for neuron cells to appear in the culture. In some embodiments, the time sufficient for neuronal cells to appear in the culture comprises at least about 1 week, at least about 2 weeks, at least about 3 weeks, at least about 4 weeks, at least about 5 weeks, at least about 6 weeks, at least about 7 weeks, or at least about 8 weeks. The multilayer population or embryoid body may be cultured in DMEM.

Neuronal cells may be isolated and further cultured to produce a population of neurons useful for transplantation. In some embodiments, isolation comprises dissociating cells or clumps of cells from the culture enzymatically, chemically, or physically and selecting neuronal cells or clumps of cells comprising neuronal cells. Embryoid bodies may be cultured in suspension and/or as an adherent culture (e.g., in suspension followed by adherent culture). In some embodiments, the embryoid bodies cultured as an adherent culture produce one or more outgrowths comprising neuronal cells. The neuronal cells have reduced HLA antigen complexity.

### IV. Treatment with Regenerative Fibroblasts and Therapeutic Energy Treatments

The present disclosure teaches that fibroblasts may be utilized together with therapeutic energy treatments for anti-inflammatory purposes, regenerative purposes, and/or cell replacement purposes. In some embodiments, there is a method of stimulating regeneration of cells and/or tissue in an individual, comprising the step of administering an extracorporeal shockwave therapy (ESWT) regimen and fibroblast cells to an anatomical area in need of regeneration. In some embodiments, the fibroblasts can be regenerative fibroblasts, which are described elsewhere herein. In some embodiments, the fibroblast cells can be cultured under conditions described elsewhere herein sufficient to differentiate the fibroblasts into regenerative fibroblast cells. In some embodiments, the regenerative fibroblast cells comprise one or more of the following biological activities: (a) inducing of angiogenesis; (b) modulating the immune system; (c) suppressing inflammation; (d) preventing of tissue atrophy;(e) regenerating of functional tissue; (f) inhibition of neuronal cell dysfunction; and/or (g) inhibition of smooth muscle degeneration. In some embodiments, the regenerative fibroblast cells are cultured under conditions described elsewhere herein sufficient to enhance the ability of the regenerative fibroblast cells to induce angiogenesis, prevent tissue atrophy, regenerate functional tissue, inhibit neuronal cell dysfunction, inhibit smooth muscle degeneration, or a combination thereof.

In one embodiment, regenerative fibroblast cells are treated with a therapeutic energy treatment in vitro. The disclosure encompasses treatment of fibroblasts in vitro with ESWT to endow fibroblasts with enhanced therapeutic activities. In some embodiments, therapeutic activities of fibroblasts include enhanced homing to area of need; for example, ESWT can stimulate expression of CXCR4, which enhances homing to hypoxic areas. In other embodiments, in vitro ESWT treatment of fibroblasts enhances production of regenerative factors by fibroblasts such as IGF-1, EGF-1, FGF1, FGF-2, VEGF, and/or angiopoietin. In other embodiments, treatment in vitro ESWT treatment of fibroblasts enhances the ability of fibroblasts to stimulate angiogenesis.

In some embodiments, the therapeutic energy treatment is electrical stimulation and/or ESWT. Without being bound by theory, these therapeutic energy treatments can increase the therapeutic efficacy of regenerative fibroblast cells by altering one or more of the following activities of regenerative cells: proliferation, differentiation, survival, cytokine and chemokine production, migration/homing to specific tissue sites, or a combination thereof.

In some embodiments, ESWT is administered by a shockwave generating device together with fibroblast cells. In some embodiments, the shockwave generating device is utilized in an aqueous environment. In some embodiments, the ESWT regimen is administered to a focal zone in an anatomical area in need thereof. In some embodiments, the focal zone comprises reduced circulation, degenerative features including atrophied and/or non-functional tissue, or a combination thereof. In some embodiments, the ESWT regimen promotes at least one or more of the following biological activities in the focal zone: (a) angiogenesis; (b) enhanced perfusion; (c) mitogenesis of smooth muscle cells; (d) augmentation of endothelial function; (e) augmentation of neurological function; and (f) reduction of inflammation.

Without wishing to be bound by theory, the application of acoustic waves creates capillary microruptures in tendon and bone. Due to microruptures the expression of growth factors such as eNOS, VEGF, PCNS and BMP is significantly increased. As a result of these processes arterioles are remodeled, stimulated to grow and new ones are formed. The new blood vessels improve blood supply and oxygenation of the treated area and support faster healing of both the tendon and the bone. Additionally, mast cell activity may be increased by using ESWT. Mast cell activation is followed by the production of chemokines and cytokines. These pro-inflammatory compounds first enhance the inflammatory process and in the next step help restore normal healing and regenerative processes, resulting in a reversal of chronic inflammation. ESWT can also accelerate procollagen synthesis, which is synthesized by smooth muscle cells and is also necessary for the repair processes of muscular structures. The therapy forces the newly created collagen fibers into a longitudinal structure that makes the newly formed tendon fibers more dense and stiff and creates a firmer structure. Further ESWT may augment neurological function by promoting cerebral angiogenesis, inhibiting molecular-cellular perturbations, and protecting white matter and neurons.

In some embodiments, a system comprising ESWT and fibroblast cells are administered together. In some embodiments, the ESWT is administered by a shockwave generating device. In some embodiments, the shockwave generating device is utilized in an aqueous environment. In some embodiments, the system is administered to a focal zone in cells or tissue. In some embodiments, the focal zone comprises reduced circulation, degenerative features, or a combination thereof. In some embodiments, degenerative features comprise atrophy or loss of function. In some embodiments, the system promotes at least one or more of the following biological activities in the focal zone: (a) angiogenesis; (b) enhanced perfusion; (c) mitogenesis of smooth muscle cells; (d) augmentation of endothelial function; (e) augmentation of neurological function; and (f) reduction of inflammation. In some embodiments, the ESWT system comprises at least one parameter selected from the group consisting of waveform parameters, treatment protocol parameters, anatomical parameters, and a combination thereof. In some embodiments, the waveform parameters comprise wave number, frequency, and/or intensity. In some embodiments, the wave intensity is about from about 50 bar to about 200 bar. In some embodiments, the wave frequency is from about 60 to about 300 shockwaves per minute. In some embodiments, the wave number is up to about 3500 per ESWT session. In some embodiments, the anatomical parameters comprise at least one focal zone to be treated. In some embodiments, the at least one focal zone comprises up to about 90% of one or more areas identified as being subject to degenerative changes in cells or tissue. In some embodiments, the degenerative changes comprise atrophy or loss of function in cells or tissue. In some embodiments, the ESWT system is combined with a drug, cellular treatment, or a combination thereof.

In some embodiments, the ESWT system comprises a treatment regimen. In some embodiments, the treatment regimen comprises ESWT and fibroblast cells administered together. In some embodiments, the ESWT is administered by a shockwave generating device. In some embodiments, the shockwave generating device is utilized in an aqueous environment. In some embodiments, the regimen is administered to a focal zone in cells or tissue. In some embodiments, the focal zone comprises reduced circulation, degenerative features, or a combination thereof. In some embodiments, degenerative features comprise atrophy or loss of function. In some embodiments, the treatment regimen promotes at least one or more of the following biological activities in the focal zone: (a) angiogenesis; (b) enhanced perfusion; (c) mitogenesis of smooth muscle cells; (d) augmentation of endothelial function; (e) augmentation of neurological function; and (f) reduction of inflammation. The ESWT treatment regimen further comprises a treatment regimen selected based on parameters including waveform parameters, treatment protocol parameters, anatomical parameters, and a combination thereof. In some embodiments, the waveform parameters comprise wave number, frequency, and intensity. In some embodiments, the wave intensity is about from about 50 bar to about 200 bar, wave frequency is from about 60 to about 300 shockwaves per minute, and wave number is up to about 3500 per ESWT session. In some embodiments, the anatomical parameters comprise at least one focal zone to be treated, which can comprise up to about 90% of one or more areas identified as being subject to degenerative changes such as atrophy or loss of function. In some embodiments, the degenerative changes comprise atrophy or loss of function in cells or tissue. In some embodiments, the ESWT regimen is combined with one or more of a drug, cellular treatment, or combinations thereof.

Without wishing to be bound by theory, it is known that under certain conditions, fibroblasts are capable of producing interleukin-1 and/or other inflammatory cytokines. In some embodiments, ESWT is administered in combination with fibroblasts that have been subjected to gene editing to prevent expression of the IL-1 gene and/or other inflammatory mediators, such as after intradiscal administration in patients with degenerative disc diseases. In some embodiments, TNF-α and inflammatory mediators are suppressed in fibroblasts administered to the brain to treat neuroinflammatory diseases such as stroke, PTSD, autism, chronic traumatic encephalopathy, suicidal ideation, anxiety, obsessions, addictions, or depression. In some embodiments, fibroblasts are pretreated with TNF-α in a manner to induce expression of growth factors and/or proliferation as described in the art and incorporated herein. Pretreatment with TNF-alpha can be performed with concentrations of TNF-alpha ranging from 1 pg/ml to 1 ug/ml, for example, 1 ng/ml to 100 ng/ml, for a time sufficient to stimulate production of grow factors at a levels at least 50% higher than baseline. Growth factors can include IGF, HGF, EGF, angiopoietin, and PDGF-1. In some embodiments, reduction in TNF-α secretion is utilized prior to, concurrently with, and/or subsequent to administration of fibroblasts, endothelial progenitor cells, stem cells, or a combination thereof.

In some embodiments, inhibitors of TNF-α secretion are selected from the group consisting of cycloheximide, auranofin, sodium aurothiomalate, triethyl gold phosphine, lipoxygenase inhibitors, ethanol, Leukotriene B4, interleukin-4, interleukin-1, polymyxin B, bile acids, interleukin-6, lactulose, oxpentifylline, mometasone, glucocorticoids, colchicine, chloroquine, FK-506, cyclosporine, phosphodiesterase inhibitors such as vinpocetine, milrinone, CI-930, rolipram, nitroquazone, zaprinast, synthetic lipid A, amrinone, N-acetylcysteine, dithiocarbamates and metal chelators, exosurf synthetic surfactant, dehydroepiandrosterone, delta-tetrahydrocannabinol, phosphatidylserine, PAF antagonist TCV-309, thalidomide, cytochrome p450 inhibitors such as Metyrapone and SKF525A, cytochalasin D, ketamine, TGF-β, interleukin-10, pentoxifylline, BRL 61,063, calcium antagonists such as dantrolene, azumolene, and diltiazem, curcumin, κ-selective opioid agonist U50,488H (trans-3,4-dichloro-N-methyl-N-[7-(1-pyrrolidinyl)cyclohexyl]benzene-acetamide methanesulfonate), alendronate, alkaloids such as fangchinoline and isotetrandrine, plant alkaloids such as tetrandrine, sulfasalazine, epinephrine, BMS-182123, adenosine, E3330, nicotine, IVIG, cardiotrophin-1, KB-R7785, CGRP, ligustrazine, dexanabinol, iloprost, activated protein C, growth hormone, spermine, FR-167653, gm-6001, estradiol, aspirin, amiodarone, and a combination thereof.

In some embodiments, inhibitors of the effects of TNF-α production are administered either systemically and/or locally, such as intradiscally to suppress inflammation and allow for enhancement of therapeutic effects of fibroblast cells and/or regenerative factors administered as disclosed herein. Other areas of local administration include areas associated with specific diseases, for example, administration into the brain or CSF for neurodegenerative diseases, administration in the liver for hepatic failure, and administration in the skin for topical diseases. Agents that inhibit TNF-α activity include but are not limited to the following: ibuprofen, indomethacin, Nedocromil sodium, cromolyn (sodium cromoglycate), spleen derived factors, pentoxifylline, 30 kDa phytoglycoprotein, NG-methyl-L-arginine, antibodies directed against core/lipid A, dexamethasone, chlorpromazine, activated α2 macroglobulin, serum amyloid A protein, neutrophil-derived proteolytic enzymes, phentolamine and propranolol, leukotriene inhibitors, nordihydroguaiaretic acid, genistein, butylated hydroxyanisole, CNI-1493, quercetin, gabexate mesylate, SM-12502, monoclonal nonspecific suppressor factor (MNSF), pyrrolidine dithiocarbamate (PDTC), aprotinin, or a combination thereof.

In one embodiment, fibroblasts are administered in combination with ESWT to increase progranulin expression and stimulate progranulin activity in the brains of patients. Without wishing to be bound by theory, it is known that decreased levels of progranulin are associated with onset of frontotemporal dementia, including in situations in which decreased levels are induced, such as in head injuries, and with Parkinson's disease. Non-genetically manipulated fibroblasts or fibroblasts genetically manipulated to enhance expression of progranulin can be administered. In one embodiment, fibroblasts are transfected with genes encoding progranulin using means known in the art and incorporated by reference. Transfection of progranulin can be performed by viral (e.g., adenoviral, lentiviral) or non-viral (e.g., electroporation, high pressure, liposomal) means. In some embodiments, fibroblasts transfected with genes encoding progranulin are further treated with ESWT to enhance progranulin activity and the efficacy of the fibroblasts.

One potential drawback for use of progranulin-transfected fibroblasts is the possibility of induced neoplasia in the fibroblasts subsequent to progranulin transfection. For example, in one study, researchers demonstrated that overexpression of the progranulin gene in SW-13 adrenal carcinoma cells and MDCK nontransformed renal epithelia resulted in the transfection-specific secretion of progranulin, acquired clonogenicity in semisolid agar, and increased mitosis in monolayer culture, whereas decreased expression of the progranulin gene impaired growth of these cells. Treatment with purified recombinant progranulin reproduced the effects of forced progranulin expression, with SW-13 and MDCK cells and other epithelia of various origins such as GPC16 colonic epithelium and A549 lung carcinoma cells exhibiting clonogenicity in soft agar and mitogenicity in monolayer culture. Progranulin overproduction in SW-13 cells also markedly increases its tumorigenicity in nude mice, demonstrating that it can regulate epithelial proliferation in vivo.

In some embodiments, transfected fibroblast cells are encapsulated to allow for release of fibroblast-derived exosomes in vivo without the fibroblasts coming into contact with other host cells to prevent neoplastic transformation. In some embodiments, the transfected fibroblasts are placed in a bioreactor to contact the host blood and allow for release of exosomes without the fibroblasts entering circulation. In some embodiments, exosomes purified from transfected fibroblasts are used therapeutically. Methods for purifying exosomes are known in the art and described herein. In some embodiments, the transfected fibroblasts are stimulated to undergo apoptosis, and the apoptotic bodies of the transfected fibroblasts are used as a therapeutic agent to induce in vivo progranulin expression in individuals suffering from frontotemporal dementia.

In some embodiments, progranulin transfection is also performed with transfection of a gene capable of inhibiting tumor formation, such as a tumor suppressor gene. Tumor suppressor genes include p53, whose transfection has been previously described and is incorporated by reference. Transfection of p53 can be performed by viral (e.g., adenoviral, lentiviral) or non-viral (e.g., electroporation, high pressure, liposomal) means. Once transfected in a manner in which p53 protein is expressed, the cells are protected from oncogenesis. In additional embodiments, agents such as suramin are administered to induce an increase in p53. Additional means for protecting against neoplasia development include suicide gene switches, used to selectively induce killing of transfected cells when desired, inducible promoters such as the ReoSwitch, or other similar “safety switch” approaches.

In one embodiment, the safety switch used to selectively induce killing of fibroblasts transfected with progranulin is the caspase 9 system in which fibroblasts are susceptible to apoptosis subsequent to administration of a compound. The caspase 9 system does not interfere with cell division or DNA synthesis and so the system is not restricted to dividing cells. Instead, the system relies on a human-derived gene, which is less likely to be immunogenic than other safety switches using, for example, a HSV-tk-derived gene. Further, the system does not involve the use of an otherwise therapeutic compound such as, for example, gancylovir. Upon toxicity of transfected fibroblasts, caspase 9 can be activated by administration of a multimeric ligand, which causes dimerization of the caspase 9 protein and induces apoptosis of the transfected fibroblasts. These features form the basis of fibroblast-based therapy, providing a safety switch following transfusion should a negative event occur.

In some embodiments, methods further comprise administering a multimeric ligand that binds to the multimeric ligand binding region of a variety of proteins. In some embodiments, the multimeric ligand binding region is contained within proteins including FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof. In some embodiments, the multimeric ligand binding region is an FKBP12 region. In some embodiments, the multimeric ligand is an FK506 dimer or a dimeric FK506 analog ligand. In some embodiments, the multimeric ligand is AP1903. Methodologies for the use of a caspase 9 system are known in the art and described, for example, in U.S. patent application Ser. No. 14/743,384.

Administration of fibroblast cells is local to an area of degeneration in some embodiments and systemic in others, depending on individual patient characteristics. administered locally into an area of degeneration or are administered systemically. In some embodiments, an area of degeneration comprises atrophy or loss of function in cells or tissues. In some embodiments, fibroblast cells are administered in a formulation with a volume of between about 0.1 ml and about 200 ml. In one embodiment, fibroblasts are concentrated in an injection solution, which may be saline, mixtures of autologous plasma together with saline, or various concentrations of albumin with saline. In some embodiments, the pH of the injection solution is from about 6.4 to about 8.3, optimally 7.4. In some embodiments, excipients such as 4.5% mannitol, 0.9% sodium chloride, or pH buffers like sodium phosphate with art-known buffer solutions are used to bring the solution to isotonicity. In some embodiments, other pharmaceutically acceptable agents including but not limited to dextrose, boric acid, sodium tartrate, propylene glycol, polyols (such as mannitol and sorbitol), and other inorganic or organic solutes are used to bring the solution to isotonicity.

In some embodiments, one or more additional therapeutic agents are administered in combination with fibroblast cells locally or systemically. In some embodiments, the one or more additional therapeutic agent is selected from the group consisting of growth factors, differentiation factors, regenerative cells, nutritional supplements, and a combination thereof. In some embodiments, the one or more additional therapeutic agent is a growth factor, stromal derived factor 1, and/or platelet concentrate. In some embodiments, the fibroblast cells are treated with one or more factors capable of stimulating smooth muscle differentiation, which are selected from the group consisting of IL-10, IL-20, IL-25, GDF-5, GDF-11, BMP-13, MIA/CD-RAP, PDGF-BB, FGF, IGF, dexamethasone, and a combination thereof.

Further, the one or more additional therapeutic agent and fibroblast cells are administered using a pharmaceutically acceptable carrier. In some embodiments, the pharmaceutically acceptable carrier is selected from a group comprising beads, microspheres, nanospheres, hydrogels, gels, polymers, ceramics, collagen, and platelet gels. In some embodiments, the one or more additional therapeutic agent is administered simultaneously with, prior to, or after administration of fibroblast cells.

In some embodiments, autologous, allogeneic, or xenogeneic fibroblast cells are administered intravenously, intramuscularly, intraperiotoneally, transdermally, intraomentally, and/or parenterally. In some embodiments, about 50 million to 500 million fibroblast cells are administered to the subject. In some embodiments, about 50 million to about 100 million fibroblast cells, about 50 million to about 200 million fibroblast cells, about 50 million to about 300 million fibroblast cells, about 50 million to about 400 million fibroblast cells, about 100 million to about 200 million fibroblast cells, about 100 million to about 300 million fibroblast cells, about 100 million to about 400 million fibroblast cells, about 100 million to about 500 million fibroblast cells, about 200 million to about 300 million fibroblast cells, about 200 million to about 400 million fibroblast cells, about 200 million to about 500 million fibroblast cells, about 300 million to about 400 million fibroblast cells, about 300 million to about 500 million fibroblast cells, about 400 million to about 500 million fibroblast cells, about 50 million fibroblast cells, about 100 million fibroblast cells, about 150 million fibroblast cells, about 200 million fibroblast cells, about 250 million fibroblast cells, about 300 million fibroblast cells, about 350 million fibroblast cells, about 400 million fibroblast cells, about 450 million fibroblast cells or about 500 million fibroblast cells may be administered to the subject. In some embodiments, fibroblast cell administration is performed as a single event, while in other embodiments, administration is performed in multiple cycles, depending on the functional result obtained and individual patient characteristics.

### V. Purification of Exosomes from Fibroblasts

In one embodiment of the present disclosure, ESWT can be used to augment the therapeutic effects of exosomes derived from fibroblasts by increasing exosome quality and/or promoting an increased concentration of therapeutic factors (e.g. growth factors) in the exosomes. Therapeutic effects include stimulation of regeneration of functional tissue by stimulating endogenous regenerative cells, angiogenesis, immune modulation, and suppression of inflammation.

In some embodiments, exosomes purified from transfected fibroblasts are used therapeutically. In some embodiments, purified fibroblast exosomes are used to decrease IL-17 production and/or inhibit pain. In some embodiments, fibroblast-derived exosomes are used to suppress inflammation, including but not limited to suppressing production of IL-1, IL-6, and TNF-alpha by macrophages. Methods for purifying exosomes are known in the art and described herein.

In one embodiment, fibroblasts are cultured using means known in the art for preserving the viability and proliferative ability of fibroblasts. Both individualized autologous exosome preparations and exosome preparations obtained from established cell lines for experimental or biological use may be used. In one embodiment, chromatography separation methods are used to prepare membrane vesicles, particularly to separate the membrane vesicles from potential biological contaminants, wherein the membrane vesicles are exosomes and cells used to generate the exosomes are fibroblast cells.

In one embodiment, strong or weak anion exchange is performed. In addition, in a specific embodiment, the chromatography is performed under pressure. Thus, in some embodiments, the chromatography consists of high performance liquid chromatography (HPLC). Different types of column supports may be used to perform the anion exchange chromatography. In some embodiments, the column supports include cellulose, poly(styrene-divinylbenzene), agarose, dextran, acrylamide, silica, ethylene glycol-methacrylate co-polymer, or mixtures thereof, e.g., agarose-dextran mixtures. Column supports include but are not limited to gels including: SOURCE™, POROS™, SEPHAROSE™, SEPHADEX™, TRISACRYL™, TSK-GEL SW™ or PW™, SUPERDEX™, TOYOPEARL HW™, and SEPHACRYL™. Therefore, in a specific embodiment, membrane vesicles, particularly exosomes, are prepared from a biological sample such as a tissue culture containing fibroblasts, comprising at least one step during which the biological sample is purified by anion exchange chromatography on an optionally-functionalized column support selected from one or more of cellulose, poly(styrene-divinylbenzene), silica, acrylamide, agarose, dextran, ethylene glycol-methacrylate co-polymer, alone or in combinations thereof.

In some embodiments, the column supports are in bead form to improve chromatographic resolution. The beads can be homogeneous and calibrated in diameter with a sufficiently high porosity to enable the penetration of objects like exosomes undergoing chromatography. The diameter of exosomes is generally between 50 and 100 nm. Thus, in some embodiments, high porosity gels with diameters between about 10 nm and about 5 μm, about 20 nm and about 2 μm, or about 100 nm and about 1 μm are used. For anion exchange chromatography, the column support used can be functionalized with a group capable of interacting with an anionic molecule. Generally, this group is composed of a ternary or quaternary amine, which defines a weak or strong anion exchanger, respectively. In some embodiments, a strong anion exchanger corresponding to a chromatography column support functionalized with quaternary amines is used. Therefore, according to a more specific embodiment, anion exchange chromatography is performed on a column support functionalized with a quaternary amine and selected from one or more of poly(styrene-divinylbenzene), acrylamide, agarose, dextran, and silica, alone or in combinations thereof, and functionalised with a quaternary amine. Column supports functionalized with a quaternary amine include but are not limited to gels including SOURCE™ Q, MONO Q™, Q SEPHAROSE™, POROS™ HQ and POROS™ QE, FRACTOGEL™ TMAE type gels and TOYOPEARL SUPER™ Q gels.

In one embodiment, the column support used to perform the anion exchange chromatography comprises poly(styrene-divinylbenzene), for example, SOURCE Q gels like SOURCE™ 15Q (Pharmacia). This column support comprises large internal pores, low resistance to liquid circulation through the gel, and rapid diffusion of exosomes to the functional groups. Biological materials including exosomes retained on the column support may be eluted using methods known in the art, for example, by passing a saline solution gradient of increasing concentration over the column support. In some embodiments, a sodium chloride solution is used in concentrations varying from 0 to 2 M, for example. Purified fractions are detected based on optical densities (OD) of the fractions measured at the column support outlet using a continuous spectrophotometric reading. In some embodiments, fractions comprising membrane vesicles are eluted at an ionic strength of approximately 350 to 700 mM, depending on vesicle type.

Different types of chromatographic columns may be used depending on experimental requirements and volumes to be purified. For example, depending on the preparations, column volumes can vary from 100 μl up to ≥10 ml, and column supports can bind and retain up to 25 mg of proteins/ml. As an example, a 100 μl column has a capacity of approximately 2.5 mg of protein, which allows for purification of approximately 2 liters of culture supernatants concentrated by a factor of 10 to 20 to yield volumes of 100 to 200 ml per preparation. Higher volumes may also be purified by increasing the column volume.

Membrane vesicles can also be purified using gel permeation liquid chromatography. In some embodiments, the anion exchange chromatography step is combined with a gel permeation chromatography step either before or after the anion exchange chromatography step. In some embodiments, the permeation chromatography step takes place after the anion exchange step. In some embodiments, the anion exchange chromatography step is replaced with the gel permeation chromatography step. To perform gel permeation chromatography, a support selected from one or more of silica, acrylamide, agarose, dextran, ethylene glycol-methacrylate co-polymer, alone or in combinations thereof, e.g., agarose-dextran mixtures. Column supports include but are not limited to gels including SUPERDEX™ 200HR (Pharmacia), TSK G6000 (TosoHaas), and SEPHACRYL™ S (Pharmacia).

Gel permeation chromatography may be applied to different biological samples. In some embodiments, biological samples include but are not limited to biological fluid from a subject (bone marrow, peripheral blood, etc.), cell culture supernatant, cell lysate, pre-purified solution, or any other composition comprising membrane vesicles. In one embodiment, the biological sample is a culture supernatant of membrane vesicle-producing fibroblast cells treated, prior to chromatography, so as to enrich the supernatant for membrane vesicles. Thus, one embodiment relates to a method of preparing membrane vesicles from a biological sample, the method characterized by at least a) an enrichment step to prepare a sample enriched with membrane vesicles and b) a purification step during which the sample is purified by anion exchange chromatography and/or gel permeation chromatography.

In some embodiments, the biological sample is composed of an enriched, pre-purified solution obtained by centrifugation, clarification, ultrafiltration, nanofiltration and/or affinity chromatography of a cell culture supernatant of a membrane vesicle-producing fibroblast cell population or biological fluid. Thus, one embodiment relates to a method of preparing membrane vesicles comprising at least the steps of a) culturing a population of membrane vesicle-producing cells under conditions enabling the release of vesicles; b) enriching membrane vesicles in the sample; and c) performing anion exchange chromatography and/or gel permeation chromatography to purify the sample.

In some embodiments, the sample (e.g. supernatant) enrichment step comprises one or more centrifugation, clarification, ultrafiltration, nanofiltration, affinity chromatography, or a combination thereof. In one embodiment, the enrichment step comprises the steps of (i) elimination of cells and/or cell debris (clarification) and (ii) concentration and/or affinity chromatography. In one embodiment, affinity chromatography following clarification is optional. In one embodiment, the enrichment step comprises the steps of (i) elimination of cells and/or cell debris (clarification); (ii) concentration; and (iii) an affinity chromatography.

In some embodiments, the elimination step of enrichment is achieved by centrifugation of the sample, for example, at a speeds below 1000 g, such as between 100 and 700 g. In one embodiment, centrifugation conditions are approximately 300 g or 600 g for a period between 1 and 15 minutes.

In some embodiments, the elimination step of enrichment is achieved by filtration of the sample. In some embodiments, sample filtration is combined with centrifugation as described. The filtration may be performed with successive filtrations using filters with a decreasing porosity. In one embodiment, filters with a porosity between 0.2 and 10 μm are used. In one embodiment, a succession of filters with a porosities of 10 μm, 1 μm, 0.5 μm, and 0.22 μm are used.

In some embodiments, the concentration step of enrichment is performed to reduce the volume of sample to be purified during chromatography. In some embodiments, the concentration step of enrichment is achieved by centrifugation of the sample at speeds between 10,000 and 100,000 g to cause the sedimentation of the membrane vesicles. In some embodiments, the concentration step of enrichment is performed as a series of differential centrifugations, with the last centrifugation performed at approximately 70,000 g. After centrifugation, the pelleted membrane may be resuspended in a smaller volume of suitable buffer.

In some embodiments, the concentration step of enrichment is achieved by ultrafiltration which allows both to concentration of the supernatant and initial purification of the vesicles. In one embodiment, the biological sample (e.g., the supernatant) is subjected to tangential ultrafiltration consisting of concentration and fractionation of the sample between two compartments (filtrate and retentate) separated by membranes of determined cut-off thresholds. Separation of the sample is carried out by applying a flow in the retentate compartment and a transmembrane pressure between the retentate compartment and the filtrate compartment. Different systems may be used to perform the ultrafiltration, such as spiral membranes (Millipore, Amicon), flat membranes, or hollow fibers (Amicon, Millipore, Sartorius, Pall, GF, Sepracor). In some embodiments, membranes with cut-off thresholds below 1000 kDa, 300 kDa to 1000 kDa, or 300 kDa to 500 kDa are used.

The affinity chromatography step can be performed in various ways, using different chromatographic support and material known in the art. In some embodiments, non-specific affinity chromatography aimed at retaining (i.e., binding) certain contaminants present within the solution without retaining the objects of interest (i.e., the exosomes) is used as a form of negative selection. In some embodiments, affinity chromatography on a dye is used, allowing for the elimination (i.e., the retention) of contaminants such as proteins and enzymes like albumin, kinases, deshydrogenases, clotting factors, interferons, lipoproteins, or also co-factors, etc. The supports used for affinity chromatography on a dye are the supports used for ion exchange chromatography functionalized with a dye. In some embodiments, the dye is selected from the group consisting of Blue SEPHAROSE™ (Pharmacia), YELLOW 86, GREEN 5, and BROWN 10 (Sigma). In some embodiments, the support is agarose. However, those of skill in the art will understand that any other support and/or dye or reactive group allowing the retention (binding) of contaminants from the biological sample to be purified can be used.

Thus, one embodiment relates to a method of preparing membrane vesicles comprising the steps of a) culturing a population of membrane vesicle-producing cells under conditions enabling release of the vesicles; b) treating the culture supernatant with at least one ultrafiltration or affinity chromatography step to produce a biological sample enriched with membrane vesicles; and c) using anion exchange chromatography and/or gel permeation chromatography to purify the biological sample. In some embodiments, step b) comprises filtration of the culture supernatant, followed by an ultrafiltration, including tangential ultrafiltration. In further embodiments, step b) comprises clarification of the culture supernatant, followed by an affinity chromatography on dye, including Blue SEPHAROSE™.

In some embodiments, after step c), the harvested material is subjected to d) one or more additional treatment and/or filtration steps for sterilization purposes. For step d), filters with a diameter ≤0.3 μm or ≤0.25 μm are used. In some embodiments, after step d), the sterilized, purified material obtained is distributed into suitable containers such as bottles, tubes, bags, syringes, etc., in a suitable storage medium and stored cold, frozen, or used extemporaneously. Thus, in some embodiments, the method of preparing membrane vesicles further comprises c) anion exchange chromatography purification of the biological sample and d) a sterilizing filtration step of the material harvested in step c). In further embodiments, the method of preparing membrane vesicles further comprises c) gel permeation chromatography purification of the biological sample and d) a sterilizing filtration step of the material harvested in step c). In additional embodiments, the method of preparing membrane vesicles further comprises c) anionic exchange purification of the biological sample followed or preceded by gel permeation chromatography and d) a sterilizing filtration step of the material harvested in step c).

### VI. Kits of the Disclosure

Any of the cellular and/or non-cellular compositions described herein or similar thereto may be comprised in a kit. In a non-limiting example, one or more reagents for use in methods for preparing fibroblasts or derivatives thereof may be comprised in a kit. Such reagents may include cells, vectors, one or more growth factors, vector(s), one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

U.S. Pat. No. 7,507,213
U.S. Patent Pub. No. 2011/0257523
U.S. Pat. No. 5,174,280
U.S. Pat. No. 5,058,569
U.S. Pat. No. 5,033,456
U.S. patent application Ser. No. 14/743,384

### EP1591070

What is claimed is:

1. A method of stimulating regeneration of cells or tissue in an individual, comprising the step of administering an extracorporeal shockwave therapy (ESWT) regimen and fibroblast cells to an anatomical area in need of regeneration in the individual.

2. The method of claim 1, wherein the fibroblasts comprise regenerative fibroblasts.

3. The method of claim 1, wherein the fibroblast cells are cultured under conditions sufficient to differentiate the fibroblasts into regenerative fibroblast cells.

4. The method of claim 2 or 3, wherein the regenerative fibroblast cells comprise one or more of the following biological activities:
(a) inducing of angiogenesis;
(b) modulating the immune system;
(c) suppressing inflammation;
(d) preventing of tissue atrophy;
(e) regenerating of functional tissue;
(f) inhibition of neuronal cell dysfunction; and
(g) inhibition of smooth muscle degeneration.

5. The method of any one of claims 2-4, wherein the regenerative fibroblast cells are cultured under conditions sufficient to enhance the ability of the regenerative fibroblast cells to induce angiogenesis, prevent tissue atrophy, regenerate functional tissue, inhibit neuronal cell dysfunction, inhibit smooth muscle degeneration, or a combination thereof.

6. The method of any one of claims 3-5, wherein the conditions comprise hypoxia.

7. The method of any one of claims 3-6, wherein the conditions further comprise treatment of the regenerative fibroblast cells with one or more growth factors, one or more differentiation factors, one or more dedifferentiation factors, or a combination thereof.

8. The method of any one of claims 2-7, wherein the regenerative fibroblast cells express one or more markers selected from the group consisting of Oct-4, Nanog, Sox-2, KLF4, c-Myc, Rex-1, GDF-3, LIF receptor, CD105, CD117, CD344, Stella, and a combination thereof.

9. The method of any one of claims 2-8, wherein the regenerative fibroblast cells do not express one or more cell surface proteins selected from the group consisting of MHC class I, MHC class II, CD45, CD13, CD49c, CD66b, CD73, CD105, CD90, and a combination thereof.

10. The method of any one of claims 2-9, wherein the regenerative fibroblast cells have enhanced GDF-11 expression compared to a control or standard.

11. The method of any one of claims 1-10, wherein the fibroblast cells are, or are derived from, fibroblasts isolated from umbilical cord, skin, cord blood, adipose tissue, hair follicle, omentum, bone marrow, peripheral blood, Wharton's Jelly, or a combination thereof.

12. The method of any one of claims 1-11, wherein the fibroblast cells are obtained from dermal fibroblasts, placental fibroblasts, adipose fibroblasts, bone marrow fibroblasts, foreskin fibroblasts, umbilical cord fibroblasts, hair follicle derived fibroblasts, nail derived fibroblasts, endometrial derived fibroblasts, keloid derived fibroblasts, or a combination thereof.

13. The method of any one of claims 1-12, wherein the fibroblast cells are autologous, allogeneic, or xenogeneic to the recipient.

14. The method of any one of claims 1-13, wherein the fibroblast cells are purified from bone marrow.

15. The method of any one of claims 1-14, wherein the fibroblast cells are purified from peripheral blood.

16. The method of any one of claims 2-15, wherein the regenerative fibroblast cells are isolated from peripheral blood of an individual who has been exposed to one or more conditions and/or one or more therapies sufficient to stimulate regenerative fibroblast cells from the individual to enter the peripheral blood of the individual.

17. The method of claim 16, wherein the conditions sufficient to stimulate regenerative fibroblast cells from the individual to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof.

18. The method of claim 16 or 17, wherein the therapies sufficient to stimulate regenerative fibroblast cells from the individual to enter the peripheral blood comprise therapies including exercise, hyperbaric oxygen, autohemotherapy by ex vivo ozonation of peripheral blood, induction of SDF-1 secretion in an anatomical area outside of the bone marrow, or a combination thereof.

19. The method of any one of claims 2-18, wherein the regenerative fibroblast cells are comprised of an enriched population of regenerative fibroblast cells.

20. The method of claim 19, wherein enrichment is achieved by:
(a) transfecting the cells with a vector comprising a fibroblast-specific promoter operably linked to a reporter or selection gene, wherein the reporter or selection gene is expressed, and
(b) enriching the population of cells for cells expressing the reporter or selection gene.

21. The method of claim 19 or 20, wherein enrichment is achieved by:
(a) treating the cells with a detectable compound, wherein the detectable compound is selectively detectable in proliferating and non-proliferating cells, and
(b) enriching the population of cells for proliferating cells.

22. The method of claim 21, wherein the detectable compound is selected from a group comprising carboxyfluorescein diacetate, succinimidyl ester, and Aldefluor.

23. The method of any one of claims 2-22, wherein the regenerative fibroblast cells are fibroblasts isolated as side population cells.

24. The method of claim 23, wherein the fibroblasts isolated as side population cells are identified based on expression of the multidrug resistance transport protein (ABCG2).

25. The method of claim 23 or 24, wherein the fibroblasts isolated as side population cells are identified based on the ability to efflux intracellular dyes.

26. The method of claims 23-25, wherein the side population cells are derived from tissues selected from the group consisting of pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, and a combination thereof.

27. The method of any of claims 1-26, wherein the fibroblast cells express CD73.

28. The method of claim 27, wherein CD73-positive fibroblast cells are derived from the group consisting of foreskin, adipose tissue, skin biopsy, bone marrow, placenta, umbilical cord, placenta, umbilical cord blood, ear lobe skin, and a combination thereof.

29. The method of claims 27 and 28, wherein the CD73-positive fibroblast cells are cultured under hypoxic conditions.

30. The method of claim 29, wherein the hypoxic conditions comprise from 0.1% oxygen to 10% oxygen for a period of 30 minutes to 3 days.

31. The method of claim 29 wherein the hypoxic conditions comprise 3% oxygen for 24 hours.

32. The method of claim 29, wherein hypoxic conditions are chemically induced.

33. The method of claim 32, wherein chemical induction of hypoxia comprises culture in cobalt (II) chloride.

34. The method of claim 33, wherein fibroblast cells are cultured with 1 μM-300 μM cobalt (II) chloride.

35. The method of claim 33, wherein the fibroblast cells are incubated with 250 μM of cobalt (II) chloride.

36. The method of claims 34 and 35, wherein the fibroblast cells are further cultured for 1-48 hours.

37. The method of claims 34 and 35, wherein the fibroblast cells are cultured for a time period of 24 hours.

38. The method of claim 29-37, wherein the hypoxic conditions induce upregulation of HIF-1α.

39. The method of claim 38, wherein expression of HIF-1α is detected by expression of VEGF secretion.

40. The method of claim 29-39, wherein the hypoxic conditions induce upregulation of CXCR4 on the fibroblast cells.

41. The method of claim 40, wherein upregulation of CXCR4 promotes homing of the fibroblast cells to an SDF-1 gradient.

42. The method of claim 27, wherein CD73-positive fibroblast cells are cultured under conditions to suppress expression of one or more apoptosis-associated genes.

43. The method of claim 42, wherein the one or more apoptosis-associated genes are selected from the group consisting of Fas, FasL, CASP1 (ICE), CASP10 (MCH4), CASP14, CASP2, CASP3, CASP4, CASP5, CASP6, CASP7, CASP8, CASP9, CFLAR (CASPER), CRADD, PYCARD (TMS1/ASC), ABL1, AKT1, BAD, BAK1, BAX, BCL2L11, BCLAF1, BID, BIK, BNIP3, BNIP3L, CASP1 (ICE), CASP10 (MCH4), CASP14, CASP2, CASP4, CASP6, CASP8, CD70 (TNFSF7), CIDEB, CRADD, FADD, FASLG (TNFSF6), HRK, LTA (TNFB), NOD1 (CARD4), PYCARD (TMS1/ASC), RIPK2, TNF, TNFRSF10A, TNFRSF10B (DR5), TNFRSF25 (DR3), TNFRSF9, TNFSF10 (TRAIL), TNFSF8, TP53, TP53BP2, TRADD, TRAF2, TRAF3, TRAF4, and a combination thereof.

44. The method of claim 42, wherein conditions to suppress expression of apoptosis-associated genes comprise administration of one or more antisense oligonucleotides.

45. The method of claim 44, wherein the antisense oligonucleotide activates RNAse H.

46. The method of claim 42, wherein conditions to suppress expression of apoptosis-associated genes comprise administration of one or more agents capable of inducing RNA interference.

47. The method of claim 46, wherein the agent comprises short interfering RNA.

48. The method of claim 46, wherein the agent comprises short hairpin RNA.

49. The method of any of claims 1-48, wherein the fibroblast cells are administered locally into an area of degeneration or are administered systemically.

50. The method of claim 49, wherein an area of degeneration comprises atrophy or loss of function in cells or tissues.

51. The method of claim 1-50, wherein the fibroblast cells are administered in a formulation with a volume of between about 0.1 ml and about 200 ml.

52. The method of any of claims 1-51, wherein one or more additional therapeutic agents are administered in combination with fibroblast cells locally or systemically.

53. The method of claim 52, wherein the one or more additional therapeutic agents are selected from the group consisting of one or more growth factors, one or more differentiation factors, regenerative cells, one or more nutritional supplements, and a combination thereof.

54. The method of claim 53, wherein the one or more additional therapeutic agents are a growth factor.

55. The method of claim 53, wherein the one or more additional therapeutic agents are stromal derived factor 1.

56. The method of claim 53, wherein the one or more therapeutic agents comprise platelet concentrate.

57. The method of claim 52-56, wherein the one or more additional therapeutic agents and the fibroblast cells are administered using a pharmaceutically acceptable carrier.

58. The method of claim 57, wherein the pharmaceutically acceptable carrier is selected from the group consisting of beads, microspheres, nanospheres, hydrogels, gels, polymers, ceramics, collagen, platelet gels, and a combination thereof.

59. The method of claim 58, wherein the carrier comprises a hydrogel.

60. The method of claim 58, wherein the carrier comprises microspheres.

61. The method of claims 52-60, wherein the one or more additional therapeutic agents are administered simultaneously with administration of fibroblast cells.

62. The method of claims 52-60, wherein the one or more additional therapeutic agents are administered prior to administration of fibroblast cells.

63. The method of claims 52-60, wherein the one or more additional therapeutic agents are administered after administration of fibroblast cells.

64. The method of claims 1-63, wherein the fibroblast cells are treated with one or more factors capable of stimulating smooth muscle differentiation.

65. The method of claim 64, wherein the factors capable of stimulating smooth muscle differentiation are selected from the group consisting of IL-10, IL-20, IL-25, GDF-5, GDF-11, BMP-13, MIA/CD-RAP, PDGF-BB, FGF, IGF, dexamethasone, and a combination thereof.

66. The method of any one of claims 1-65, wherein ESWT is administered by a shockwave generating device together with the fibroblast cells.

67. The method of claim 66, wherein the shockwave generating device is utilized in an aqueous environment.

68. The method of claim 1-67, wherein the ESWT regimen is administered to a focal zone in cells or tissue.

69. The method of claim 68, wherein the focal zone comprises reduced circulation, degenerative features, or a combination thereof.

70. The method of claim 69, wherein degenerative features comprise atrophy or loss of function.

71. The method of claims 1-70, wherein the ESWT regimen promotes at least one or more of the following biological activities in the focal zone:
(a) inducing of angiogenesis;
(b) modulating the immune system;
(c) suppressing inflammation;
(d) preventing of tissue atrophy;
(e) regenerating of functional tissue;
(f) inhibition of neuronal cell dysfunction; and
(g) inhibition of smooth muscle degeneration.

72. The method of claims 1-71, wherein the ESWT regimen comprises a treatment regimen selected based on at least one parameter selected from the group consisting of waveform parameters, treatment protocol parameters, anatomical parameters, and a combination thereof.

73. The method of claim 72, wherein the waveform parameters comprise wave number, frequency, and intensity.

74. The method of claim 73, wherein the wave intensity is about from about 50 bar to about 200 bar.

75. The method of claim 73, wherein the wave frequency is from about 60 to about 300 shockwaves per minute.

76. The method of claim 73, wherein the wave number is up to about 3500 per ESWT session.

77. The method of claim 72, wherein the anatomical parameters comprise at least one focal zone to be treated.

78. The method of claim 77, wherein the at least one focal zone comprises up to about 90% of one or more areas identified as being subject to degenerative changes in cells or tissue.

79. The method of claim 78, wherein the degenerative changes comprise atrophy or loss of function in cells or tissue.

80. The method of any of claims 1-79, further comprising combining the ESWT regimen with a drug, cellular treatment, or a combination thereof.

81. The method of claim 80, wherein the cellular treatment comprises mesenchymal stem cells, hematopoietic stem cells, or embryonic-like stem cells.

82. A system comprising ESWT and fibroblast cells.

83. The system of claim 82, wherein the ESWT is administered by a shockwave generating device.

84. The system of claim 83, wherein the shockwave generating device is utilized in an aqueous environment.

85. The system of claims 82-84, wherein the system is administered to a focal zone in cells or tissue.

86. The system of claim 85, wherein the focal zone comprises reduced circulation, degenerative features, or a combination thereof.

87. The system of claim 86, wherein degenerative features comprise atrophy or loss of function.

88. The system of claims 82-87, wherein the system promotes at least one or more of the following biological activities in the focal zone:
(a) inducing of angiogenesis;
(b) modulating the immune system;
(c) suppressing inflammation;
(d) preventing of tissue atrophy;
(e) regenerating of functional tissue;
(f) inhibition of neuronal cell dysfunction; and
(g) inhibition of smooth muscle degeneration.

89. The system of claims 82-88, wherein the ESWT system comprises at least one parameter selected from the group consisting of waveform parameters, treatment protocol parameters, anatomical parameters, and a combination thereof.

90. The system of claim 89, wherein the waveform parameters comprise wave number, frequency, and intensity.

91. The system of claim 90, wherein the wave intensity is about from about 50 bar to about 200 bar.

92. The system of claim 90, wherein the wave frequency is from about 60 to about 300 shockwaves per minute.

93. The system of claim 90, wherein the wave number is up to about 3500 per ESWT session.

94. The system of claim 89, wherein the anatomical parameters comprise at least one focal zone to be treated.

95. The system of claim 94, wherein the at least one focal zone comprises up to about 90% of one or more areas identified as being subject to degenerative changes in cells or tissue.

96. The system of claim 95, wherein the degenerative changes comprise atrophy or loss of function in cells or tissue.

97. The system of claims 82-96, further comprising combining the ESWT system with a drug, cellular treatment, or a combination thereof.

98. The system of claim 97, wherein the cellular treatment comprises mesenchymal stem cells, hematopoietic stem cells, or embryonic-like stem cells.

99. The system of any one of claims 82-98, wherein the fibroblast cells express one or more markers selected from the group consisting of Oct-4, Nanog, Sox-2, KLF4, c-Myc, Rex-1, GDF-3, LIF receptor, CD105, CD117, CD344, Stella, and a combination thereof.

100. The system of any one of claims 82-99, wherein the fibroblast cells do not express one or more cell surface proteins selected from the group consisting of MHC class I, MHC class II, CD45, CD13, CD49c, CD66b, CD73, CD105, CD90, and a combination thereof.

101. The system of any one of claims 82-100, wherein the fibroblast cells have enhanced GDF-11 expression compared to a control or standard.

102. The system of any one of claims 82-101, wherein the fibroblast cells are, or are derived from, fibroblasts isolated from umbilical cord, skin, cord blood, adipose tissue, hair follicle, omentum, bone marrow, peripheral blood, Wharton's Jelly, or a combination thereof.

103. The system of any one of claims 82-102, wherein the fibroblast cells are obtained from dermal fibroblasts, placental fibroblasts, adipose fibroblasts, bone marrow fibroblasts, foreskin fibroblasts, umbilical cord fibroblasts, hair follicle derived fibroblasts, nail derived fibroblasts, endometrial derived fibroblasts, keloid derived fibroblasts, or a combination thereof.

104. The system of any one of claims 82-103, wherein the fibroblast cells are autologous, allogeneic, or xenogeneic to the recipient.

105. The system of any one of claims 82-104, wherein the fibroblast cells are purified from bone marrow.

106. The system of any one of claims 82-104, wherein the fibroblast cells are purified from peripheral blood.

107. The system of any one of claims 82-104, wherein the fibroblast cells are isolated from peripheral blood of an individual who has been exposed to one or more conditions and/or one or more therapies sufficient to stimulate regenerative fibroblast cells from the individual to enter the peripheral blood of the individual.
